SEQUENCE LISTING (11 sequences)

SEQ ID NO: 1 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  source 1..29: mol_type = other DNA
  modified_base 23: mod_base = OTHER; note = alkene-thymidine
aatgatacggcgaccaccgaganctacac

SEQ ID NO: 3 - DNA, 29 bp, synthetic construct
  source 1..29: mol_type = other DNA
  modified_base 23: mod_base = i
aatgatacggcgaccaccgaganctacac

SEQ ID NO: 4 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 7 - DNA, 29 bp, Synthetic construct
  source 1..29: mol_type = other DNA
  modified_base 23: mod_base = OTHER; note = allyl-thymine
aatgatacggcgaccaccgaganctacac

SEQ ID NO: 8 - DNA, 35 bp, Synthetic construct
  source 1..35: mol_type = other DNA
gctggcacgtccgaacgcttcgttaatccgttgag

SEQ ID NO: 9 - DNA, 35 bp, Synthetic construct
  source 1..35: mol_type = other DNA
cgtcgtctgccatggcgcttcggtggatatgaact

SEQ ID NO: 10 - DNA, 35 bp, Synthetic construct
  source 1..35: mol_type = other DNA
acggccgctaatatcaacgcgtcgaatccgcaact

SEQ ID NO: 11 - DNA, 34 bp, Synthetic construct
  source 1..34: mol_type = other DNA
gccgcgttacgttagccggactattcgatgcagc

(19) United States
(12) Patent Application Publication
(10) Pub. No.: US 2025/0188444 A1
(43) Pub. Date: Jun. 12, 2025

(54) FLOW CELLS AND PHOTODEGRADABLE OLIGONUCLEOTIDE BLOCKERS

(71) Applicant: ILLUMINA, INC., San Diego, CA (US)

(72) Inventors: Gabriele Canzi, San Diego, CA (US); Michael Neville, San Diego, CA (US)

(21) Appl. No.: 18/970,109

(22) Filed: Dec. 5, 2024

Related U.S. Application Data
(60) Provisional application No. 63/607,428, filed on Dec. 7, 2023.

(51) Int. Cl. C12N 15/10

(52) U.S. Cl. CPC C12N 15/1065

(57) ABSTRACT

An example of a flow cell includes a substrate; a polymeric hydrogel applied over at least a portion of a surface of the substrate; and a plurality of primers attached to the polymeric hydrogel. The flow cell further includes a plurality of photodegradable oligonucleotide blockers respectively hybridized to at least some of the plurality of primers.

### CROSS-REFERENCE TO RELATED APPLICATIONS

This application claims the benefit of U.S. Provisional Application Ser. No. 63/607,428, filed Dec. 7, 2023, the contents of which is incorporated by reference herein in its entirety.

### REFERENCE TO SEQUENCE LISTING

The instant application contains a Sequence Listing, which has been submitted electronically in XML file format and is hereby incorporated by reference in its entirety. Said XML copy, created on Dec. 18, 2024 is named IL1272B_IP-2705-US_Revised_Sequence_Listing.xml and is 14,952 bytes in size.

### BACKGROUND

Various protocols in biological or chemical research involve performing a large number of controlled reactions on local support surfaces or within predefined reaction chambers of a flow cell. The reactions may then be observed or detected, and subsequent analysis may help identify or reveal properties of molecules involved in the controlled reactions. In some examples, the reactions generate fluorescence, and thus an optical system that is configured for fluorescence detection may be used to analyze the controlled reactions. In other examples, the controlled reactions alter charge, conductivity, or some other electrical property of the molecule(s) being analyzed, and thus an electronic system may be used for detection.

### SUMMARY

Some biotechnological applications utilize a substrate having a polymer coating thereon, where the polymer-coated substrate surface is used for the preparation and/or analysis of biological molecules. Molecular analyses, such as certain nucleic acid sequencing methods, may operate using nucleic acid strands (e.g., primers) that are attached to the polymer-coated surface. In such nucleic acid sequencing methods, the primers are capable of seeding DNA library templates thereto. In some instances, however, it may be desirable to temporarily block the primers that are positioned within a given portion of the polymer-coated substrate, such that the blocked primers are rendered temporarily incapable of seeding molecules (e.g., the DNA library templates) thereto.

Disclosed herein are flow cells and methods utilizing photodegradable oligonucleotide blockers. The oligonucleotide blockers respectively hybridize (e.g., base-pair or hydrogen bond) with primers attached to the polymer-coated substrate surface, thereby passivating the hybridized primers. Upon selective exposure to a preselected wavelength of light, the oligonucleotide blockers undergo a structural change and become removable (or are removed) from the passivated primers, thereby rendering those primers chemically active and available for seeding a target molecule. As such, passivated primers within predetermined regions of the polymer-coated surface can be selectively de-blocked (and subsequently seeded with library templates) at desired times. In some examples described herein, the exposure/seeding of the passivated primers within predetermined regions is performed such that individual regions become respectively seeded with library templates from different DNA libraries. As such, using the photodegradable oligonucleotide blockers disclosed herein, library templates from different DNA libraries can be selectively and respectively seeded to (de-blocked) primers within discrete regions of a polymer-coated substrate surface.

Accordingly, the photodegradable oligonucleotide blockers disclosed herein may introduce versatility to the polymer-coated substrate surface by enabling the selective seeding of library templates from different DNA libraries within different regions of a single polymer-coated substrate.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of an example flow cell;

FIG. 1B is an enlarged, and partially cutaway cross-sectional, perspective view of an example of a lane defined in a substrate of the flow cell;

FIG. 1C is an enlarged, and partially cutaway cross-sectional, perspective view of an example of an architecture within a flow channel of the flow cell that includes depressions;

FIG. 1D is an enlarged, and partially cutaway cross-sectional, perspective view of an example of another architecture within a flow channel of the flow cell that includes functionalized pads;

FIG. 2 depicts a chemical structure of an example of a photodegradable moiety and a chemical reaction involving the photodegradable moiety;

FIG. 3 is a schematic illustration of a flow cell including a complementary metal-oxide semiconductor (CMOS) imaging device that is coupled to a substrate of the flow cell;

FIG. 4 schematically illustrates two example methods (A., B., C. and A., D., E., F.) utilizing a pre-grafted polymeric hydrogel, where:
A. illustrates a lane that has been defined in a substrate, B. illustrates the application of a polymeric hydrogel that is pre-grafted with a plurality of double-stranded entities over the structure of A. and within the lane, and C. illustrates the removal of the polymeric hydrogel and the double-stranded entities from portions of the structure of B.;
A. illustrates a lane that has been defined in a substrate, D. illustrates the application of a polymeric hydrogel that is pre-grafted with a plurality of primers over the structure of A. and within the lane, E. illustrates the removal of the polymeric hydrogel and the primers attached thereto from portions of the structure of E., and F. illustrates the respective hybridization of each of a plurality of photodegradable oligonucleotide blockers to individual primers within the lane;

FIG. 5 schematically illustrates two example methods (A., B., C., D., E. and A., B., C., E) utilizing a non-pre-grafted polymeric hydrogel, where:
A. illustrates a lane that has been defined in a substrate, B. illustrates the application of a polymeric hydrogel over the structure of A. and within the lane, C. illustrates the removal of the polymeric hydrogel from portions of the structure of B., D. illustrates a plurality of primers grafted to the polymeric hydrogel within the lane, and E. illustrates the respective hybridization of each of a plurality of photodegradable oligonucleotide blockers to individual primers within the lane;
A. illustrates a lane that has been defined in a substrate, B. illustrates the application of a polymeric hydrogel over the structure of A. and within the lane, C. illustrates the removal of the polymeric hydrogel from portions of the structure of B., and E. illustrates a plurality of double-stranded entities grafted to the polymeric hydrogel within the lane; and

FIG. 6A through FIG. 6D illustrate a method of using a flow cell including primers that are hybridized to photodegradable oligonucleotide blockers, where FIG. 6A depicts two sub-sets of primers respectively positioned at different areas of a lane defined in the substrate of the flow cell, wherein each of the primers in each of the two sub-sets has a first photodegradable oligonucleotide blocker hybridized thereto, FIG. 6B depicts exposing a first of the two sub-sets of primers to a preselected light wavelength and the resultant dehybridization/removal of the photodegradable oligonucleotide blockers from the primers in the first sub-set, FIG. 6C depicts seeding of DNA library templates to at least one of the de-blocked primers in the first sub-set, whereby at least one of the de-blocked primers in the first sub-set remains unseeded with DNA library templates, and FIG. 6D depicts the hybridization of a second photodegradable oligonucleotide blocker to the at least one unseeded, de-blocked primer in the first sub-set.

### DEFINITIONS

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, adjacent, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

An “acrylamide monomer” refers to a monomer with the structure

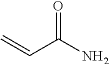

or a monomer including an acrylamide group. Examples of the monomer including an acrylamide group include azido acetamido pentyl acrylamide:

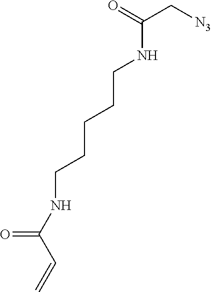 [SMILES: C=CC(=O)NCCCCCNC(=O)CN=[N+]=[N-]]

### and N-isopropylacrylamide:

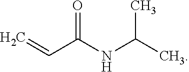

Other acrylamide monomers may be used.

The term “activation,” as used herein, refers to a process that generates reactive groups at the surface of a single layer substrate or an outermost layer of a multi-layer substrate. Activation may be accomplished, for example, using silanization or plasma ashing. Though not explicitly shown in the figures, when activation of a surface is performed, it is to be understood that silane groups or —OH functional groups become introduced to the surface. These functional groups can then be used to covalently attach a material, such as a polymeric hydrogel, to the surface that includes the functional groups.

An “aldehyde,” refers to an organic compound containing a functional group with the structure —CHO, which includes a carbonyl center (i.e., a carbon double-bonded to oxygen), with the carbon atom also being bonded to hydrogen and an R group (such as an alkyl or other side chain). The general structure of an aldehyde is:

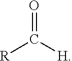

An “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and tert-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen

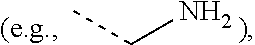

C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, the terms “attach,” “attached,” and “attachment” refer to the state of two things being joined, fastened, adhered, connected or bound to each other, either directly or indirectly. The terms may refer to chemical attachment or physical attachment. As examples of chemical attachment, a nucleic acid can be attached to a polymeric hydrogel by a covalent or non-covalent bond, or a photodegradable oligonucleotide blocker can be attached (e.g., hybridized) to nucleotides of a primer strand via hydrogen bonding. As an example of physical attachment, in enclosed versions of the flow cell disclosed herein, a lid may be physically coupled to a patterned structure at a bonding region (e.g., using an adhesive).

An “azide” or “azido” functional group refers to —N₃.

As used herein, a “bonding region” refers to an area of a structure that is to be bonded to another material, which may be, as examples, a spacer layer, a lid, another structure, etc., or combinations thereof (e.g., a spacer layer and a lid, or a spacer layer and another structure). The bond that is formed at the bonding region may be a chemical bond (as described above), or a mechanical bond (e.g., using a fastener, etc.). The bonding region may be free of surface chemistry (e.g., may be free of polymeric hydrogel, of primers of a primer set, and of photodegradable oligonucleotide blockers).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” refers to —COOH.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in the ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties or attachment of one substance to another. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating/deposition, aerosol printing, screen printing, microcontact printing, inkjet printing, or the like.

As used herein, the term “depression” refers to a discrete concave feature defined in a substrate and having a surface opening. In some instances, the surface opening is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” as used herein refers to

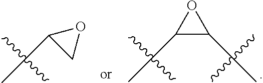

As used herein, the term “flow cell” is intended to refer to a vessel having an enclosed or open flow channel where a reaction can be carried out. A flow cell with an enclosed channel may also include an inlet for delivering reagent(s) to the channel and an outlet for removing reagent(s) from the channel. In some examples, the flow cell enables the detection of the reaction that occurs therein. For example, the flow cell can include one or more transparent surfaces allowing for the optical detection of arrays, optically labeled molecules, or the like. As another example, the flow cell can include a complementary metal oxide semiconductor chip coupled thereto, allowing for the electrical detection of arrays, optically labeled molecules, or the like.

As used herein, a “flow channel” or “channel” may be (i) an area defined between two bonded components or may be (ii) a concave or recessed area, or lane, defined in a single substrate. In either case, the “flow channel” or “channel” can selectively receive a liquid sample, reagents, etc. In some examples, the flow channel may be defined between two substrates, and thus the flow channel may be enclosed and in fluid communication with surface chemistry disposed on either of the two substrates. In other examples, the flow channel may be defined between one substrate and a lid, and thus the flow channel may be enclosed and in fluid communication with surface chemistry disposed on the one substrate. In still other examples, the flow channel may be defined by a concave or recessed area that is formed in a surface of a single substrate, and thus the flow channel may be in fluid communication with surface chemistry within the concave or recessed area. In these examples, the surface chemistry within the concave or recessed area is open to the surrounding environment.

As used herein, a “functionalized pad” or “pad” refers to a polymeric hydrogel applied on a substrate surface and at least one primer attached thereto. Each functionalized pad, when included, is discrete from other functionalized pads and is surrounded by interstitial regions, as defined herein.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

The term “hydrazone” or “hydrazonyl,” as used herein, refers to a

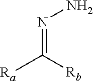

group, in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

“Hybridization” or “hybridize(d),” as used herein, refers to base-pairing of nucleotides (i.e., hydrogen bonding). For example, an oligonucleotide blocker may be hybridized to a primer (of a primer set) via base pairing of (i) nucleotides in the backbone of the nucleotide-based blocker and (ii) nucleotides of the primer.

The term “hydrogel” or “polymeric hydrogel” refers to a semi-rigid polymer that is permeable to liquids and/or gases. The hydrogel can swell when liquid (e.g., water) is taken up and can contract when liquid is removed, e.g., by drying. While a hydrogel may absorb water, the examples described herein are not water-soluble. In some examples, the polymeric hydrogel is referred to as being a “pre-grafted polymeric hydrogel,” meaning that primers (of a primer set) are grafted/attached to the polymeric hydrogel before the polymeric hydrogel is deposited over a substrate. In some of these examples, i.e., when the primers are hybridized to photodegradable oligonucleotide blockers before the pre-grafted hydrogel is deposited over a substrate, the hydrogel may further be referred to as a “pre-grafted, pre-hybridized polymeric hydrogel.” In other examples, the polymeric hydrogel is referred to as being a “non-pre-grafted polymeric hydrogel,” meaning that primers (of a primer set) are grafted/attached to the polymeric hydrogel after the polymeric hydrogel is deposited over the substrate.

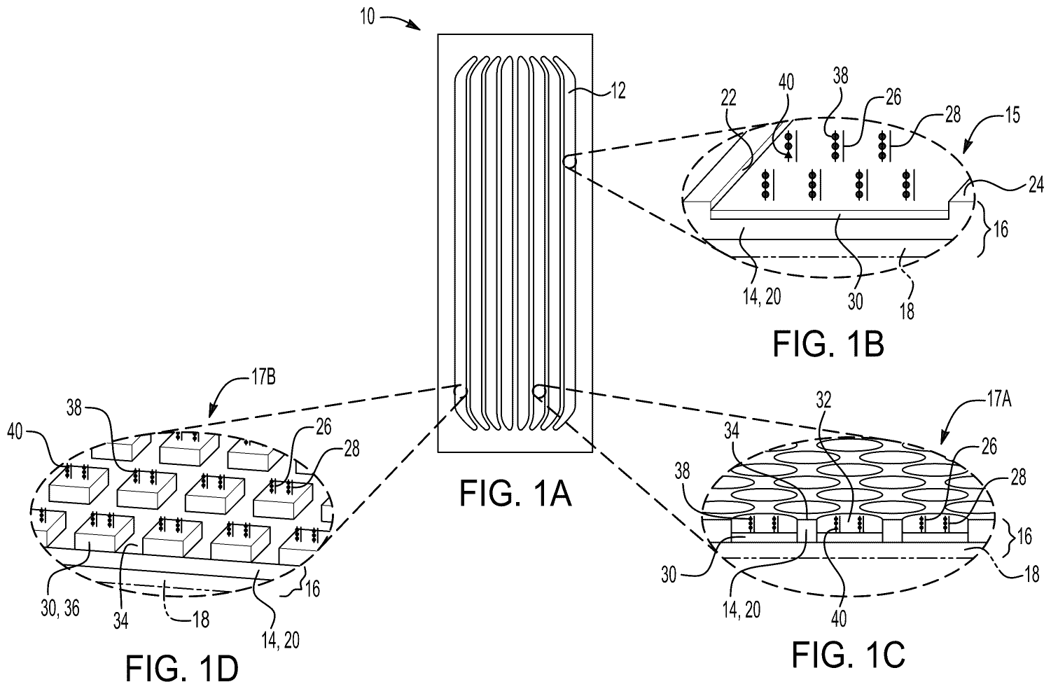

As used herein, the term “interstitial region” refers to an area, e.g., of a substrate, that separates individual depressions from one another (see interstitial regions 34 separating depressions 32 in FIG. 1C) or that separates individual functionalized pads from one another (see interstitial regions 34 separating functionalized pads 36 in FIG. 1D). The separation provided by an interstitial region can be partial or full separation.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)=NOH]or from the reaction between hydroxylamine and an aldehyde.

“Nitrone,” as used herein, means a

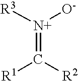

group in which R¹, R², and R³ may be any of the Rₐ and R_{b} groups defined herein, except that R³ is not hydrogen (H).

As used herein, a “nucleotide” includes a nitrogen-containing heterocyclic base, a sugar, and one or more phosphate groups. Nucleotides are monomeric units of a nucleic acid sequence. In RNA (ribonucleic acid), the sugar is a ribose, and in DNA (deoxyribonucleic acid), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). As used herein, nucleotides may be a component of primers (of a primer set), or nucleotides may be a component of a nucleotide-based backbone of a photodegradable oligonucleotide blocker.

In some examples, the term “over” may mean that one component or material is positioned directly on another component or material. When one is directly on another, the two are in physical contact with each other. In FIG. 1B, for example, when a multi-layer substrate 16 (which includes a base support 18 having an additional layer 20 positioned thereon) is used, the layer 20 is positioned directly “over” the base support 18, such that there is no intervening component or material therebetween.

In other examples, the term “over” may mean that one component or material is positioned indirectly on another component or material. By indirectly on, it is meant that a gap or an additional component or material is positioned between the two components or materials. In FIG. 1B, for example, when the multi-layer substrate 16 is utilized, the polymeric hydrogel 30 is indirectly over the base support 18. The layer 20 is positioned therebetween.

A “patterned structure” refers to a substrate that has been patterned with depressions (see the patterned structure 17A including depressions 32 in FIG. 1C) or with functionalized pads (see the patterned structure 17B including functionalized pads 36 in FIG. 1D). In some examples, the substrate is exposed to patterning techniques (e.g., etching, nanoimprint lithography, photolithography, etc.) in order to generate the desired pattern(s). However, the term “patterned structure” is not intended to imply that such patterning techniques have to be used to generate the pattern. In contrast, an “unpatterned structure” refers to a substrate having a recessed or concave feature e.g., a lane) defined therein (in which surface chemistry is included), but that does not include functionalized pads or depressions within the recessed or concave feature (see the unpatterned structure 15 including the lane 22 in FIG. 1B).

The terms “photodegradable oligonucleotide blocker,” “oligonucleotide blocker,” “photodegradable blocker,” and “blocker,” as used herein, are interchangeable and refer to a nucleotide-based backbone (e.g., a chain of individual nucleotides) having one or more units of a photodegradable moiety interspersed throughout the backbone as linking molecules. The blocker is capable of hybridizing to and passivating a target string of nucleotides, such as an oligonucleotide primer, thereby rendering the oligonucleotide primer temporarily incapable of seeding templates from a DNA library thereto. The “photodegradable moiety” included in the nucleotide-based backbone of the blocker refers to a chemical structure that is capable of undergoing a chemical transformation upon exposure to a predetermined wavelength of light. The transformation undergone by the photodegradable moiety enables a portion of the oligonucleotide blocker (or the entire oligonucleotide blocker) to be removed from a primer to which the oligonucleotide blocker is hybridized. In some examples described herein, the photodegradable moiety is an ortho-nitrobenzyl containing moiety (e.g., a benzyl ring having an attached nitro (N₂O) group and an additional substituent in an ortho configuration (i.e., the nitro group and the additional substituent are attached to directly adjacent carbons in the benzyl ring). Further examples of photodegradable moieties are described herein.

When a substance or moiety is said to be “photodegradable” herein, it is meant that the substance or moiety undergoes a desired chemical transformation upon exposure to a preselected wavelength of light. As examples, upon exposure to the preselected wavelength of light, the photodegradable materials described herein may undergo a change in solubility, or hybridization state, or binding state, or structural connectivity, etc.

As used herein, the term “polyhedral oligomeric silsesquioxane” (an example of which is commercially available under the tradename “POSS®” from Hybrid Plastics) refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS® include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

As used herein, a “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymeric hydrogel. The nucleotides that make up the primer are capable of hybridizing (i.e., base-pairing) to/with the nucleotides of a photodegradable oligonucleotide blocker. The primer length can be any number of bases long and can include a variety of non-natural nucleotides. In an example, the sequencing primer ranges from 10 to 150 bases, or from 10 to 60 bases, or from 20 to 40 bases, etc.

A “spacer layer,” as used herein refers to a material that bonds two components together. In some examples, the spacer layer can be a radiation-absorbing material that aids in bonding, or can be put into contact with a radiation-absorbing material that aids in bonding.

The term “substrate” may be used herein in conjunction with the term “single layer substrate” or “multi-layer substrate.” A single layer substrate is one layer of a support material that can be imprinted or otherwise processed to form a lane (see the substrate 14 shown in FIG. 1B) and/or that can be patterned with depressions (see FIG. 1C) or functionalized pads (see FIG. 1D). The multi-layer substrate includes at least two layers, e.g., a base support 18 with an additional layer 20 thereon (see FIG. 1B), the latter of which can be imprinted or otherwise processed to form the lane and/or that can be patterned with depressions (see FIG. 1C) or functionalized pads (see FIG. 1D).

“Surface chemistry,” as defined herein, refers to a polymeric hydrogel and at least one primer attached thereto. In some instances, the term further refers to a plurality of photodegradable oligonucleotide blockers, i.e., when these blockers are individually hybridized to respective primers. Surface chemistry may be disposed within a lane defined in a substrate surface (see the lane 22 in FIG. 1B), or may be disposed within depressions defined in a substrate surface (see the depressions 32 in FIG. 1C), or may form functionalized pads on a substrate surface (see the functionalized pads 36 in FIG. 1D).

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refers to five-membered heterocyclic group including four nitrogen atoms. Tetrazole can be optionally substituted.

The term “transparent” when describing a material (e.g., substrate, layer, etc.) means that that the material allows light of a particular wavelength or range of wavelengths to pass through. Transparency may be quantified using transmittance, i.e., the ratio of light energy falling on a body to that transmitted through the body. The transmittance of a transparent material will depend upon the thickness of the material and the wavelength of light. In the examples disclosed herein, the transmittance of the transparent material may range from 0.25 (25%) to 1 (100%). The material may be a pure material, a material with some impurities, or a mixture of materials, as long as the resulting material is capable of the desired transmittance. As an example, tantalum pentoxide (i.e., the inorganic compound with the formula Ta₂O₅) is transparent, having a transmittance ranging from about 0.25 (25%) to 1 (100%), to wavelengths ranging from about 0.35 μm (350 nm) to at least 1.8 μm (1800 nm). Additionally, depending upon the transmittance of the material, the time for light exposure and/or the output power of the light source may be increased or decreased to deliver a suitable dose of light energy through the transparent material to achieve the desired effect.

### DETAILED DESCRIPTION

Disclosed herein are flow cells that include and methods that utilize photodegradable oligonucleotide blockers. Examples of the flow cells and formation thereof are described in reference to FIG. 1A through FIG. 5 and an example of the method for using the flow cells is described in reference to FIG. 6.

### Flow Cells and Flow Cell Formation

Examples of the flow cells disclosed herein generally include a substrate; a polymeric hydrogel applied over at least a portion of a surface of the substrate; a plurality of primers attached to the polymeric hydrogel; and a plurality of photodegradable oligonucleotide blockers respectively hybridized to at least some of the plurality of primers.

FIG. 1A depicts an example of the flow cell 10 from a top view. The flow cell 10 shown in FIG. 1A may include patterned structure(s), unpatterned structure(s), and/or a lid. An example of an unpatterned structure 15 including a lane 22 defined therein is shown in FIG. 1B, and different examples of patterned structures 17A, 17B that respectively include depressions 32 and functionalized pads 36 are separately shown in FIG. 1C and in FIG. 1D.

Enclosed examples of the flow cell 10 disclosed herein may include one unpatterned structure 15 or one patterned structure 17A, 17B bonded to a lid (lid not shown in FIG. 1B through FIG. 1D), e.g., at a bonding region 24 (see FIG. 1B). Enclosed examples of the flow cell 10 may alternatively include one (un)patterned structure 15, 17A, 17B bonded to another unpatterned or patterned structure via a spacer layer at the bonding region 24 (second structure and spacer layer not shown). Open-wafer examples of the flow cell 10 include a single unpatterned structure 15 or patterned structure 17A, 17B, where the surface chemistry included in the single unpatterned structure 15 or patterned structure 17A, 17B is open to a surrounding environment.

In enclosed versions of the flow cell 10, the spacer layer used to attach the unpatterned structure 15 or patterned structure 17A, 17B to the lid may be any material that will seal portions of the unpatterned structure 15 or patterned structure 17A, 17B and the lid. Alternatively, the spacer layer may be any material that will seal portions of the unpatterned structure 15 or patterned structure 17A, 17B and the second unpatterned or patterned structure. As examples, the spacer layer may be an adhesive, a radiation-absorbing material that aids in bonding, or the like. In some examples, the spacer layer is the radiation-absorbing material, e.g., KAPTON® black (DuPont de Nemours, Inc.).

In both enclosed and open-wafer versions of the flow cell 10, the unpatterned structure 15 or patterned structure 17A, 17B of the flow cell 10 may be a single layer substrate 14. Alternatively, the unpatterned structure 15 or patterned structure 17A, 17B may be a multi-layer substrate 16 including a base support 18 having a layer 20 positioned thereon. The single layer substrate 14 and the multi-layer substrate 16 are depicted in each of FIG. 1B, FIG. 1C, and FIG. 1D.

Examples of suitable materials for the substrate 14 include siloxanes (e.g., epoxy siloxane), glass, modified or functionalized glass, polymeric materials (including acrylics, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.), ceramics/ceramic oxides, silica (i.e., silicon dioxide (SiO₂)), fused silica, or silica-based materials, aluminum silicate, silicon and modified silicon (e.g., boron doped p+silicon), silicon nitride (Si₃N₄), tantalum pentoxide (Ta₂O₅) or other tantalum oxide(s) (TaOₓ), hafnium oxide (HfO₂), carbon, metals, resins, or the like. Examples of suitable resins include inorganic oxides, such as tantalum pentoxide (e.g., Ta₂O₅) or other tantalum oxide(s) (TaOₓ), aluminum oxide (e.g., Al₂O₃), silicon oxide (e.g., SiO₂), hafnium oxide (e.g., HfO₂), indium tin oxide, titanium dioxide, etc., or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. It is to be understood that the material of the substrate 14 may be any material that can be etched, imprinted, or manipulated to form the lane 22 shown in FIG. 1B, or to form the depressions 32 shown in FIG. 1C. The material of the substrate 14 may further be any suitable material that can be patterned with the functionalized pads 36 shown in FIG. 1D.

As mentioned, examples of the multi-layer substrate 16 include the base support 18 and at least one other layer 20 positioned thereon. Any example of the material of the single layer substrate 14 provided herein may be used as the material for the base support 18 of the multi-layer substrate 16. Examples of suitable materials for the layer 20 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. It is to be understood that in examples of the flow cell 10 that include the substrate 16, the other layer 20 (positioned on the base support 18) may be any material that can be etched, imprinted, or manipulated to form the lane 22 shown in FIG. 1B, or to form the depressions 32 shown in FIG. 1C. The material of the layer 20 may further be any material that can be patterned with the functionalized pads 36 shown in FIG. 1D.

Suitable deposition techniques for the material of the substrate 14 or for the material(s) of the components of the substrate 16 (i.e., the base support 18 and the layer 20) include chemical vapor deposition, dip coating, dunk coating, spin coating, spray coating, puddle dispensing, ultrasonic spray coating, doctor blade coating, aerosol printing, screen printing, microcontact printing, etc. It is to be understood that the deposition technique(s) that is/are used may depend, in part, upon the material of the substrate 14 or the material of the components of the substrate 16.

The single layer substrate 14 or the base support 18 (of the multi-layer substrate 16) may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a substrate 14 or base support 18 with any suitable dimensions may be used.

The thickness of the layer 20 (when the substrate 16 is used) is variable. In examples of the flow cell 10 that include depressions 32 (as in FIG. 1C), the thickness of the layer 20 is greater than the desired depth for the depressions 32 formed therein. In examples of the flow cell 10 that include the lane 22 (as in FIG. 1B), the thickness of the layer 20 is greater than the desired depth for the lane 22 formed therein.

Suitable patterning techniques for the substrate 14 (or for the layer 20 of the substrate 16) include photolithography, nanoimprint lithography (NIL), stamping techniques, embossing techniques, molding techniques, microetching techniques, etc. It is to be understood that the patterning technique(s) that is/are used may depend, in part, upon the material used for the substrate 14 or for the layer 20 of the substrate 16.

Regardless of whether the substrate 14 or 16 is used (and regardless of the patterned structure 17A, 17B or unpatterned structure 15 that is used), the enclosed flow cell 10 and the open-wafer flow cell 10 may include one or more flow channel(s) 12. In the enclosed flow cell 10, the flow channel(s) 12 is/are defined between the one (un)patterned structure 15, 17A, 17B and the lid (not shown) or between the one (un)patterned structure 15, 17A, 17B and the second (un)patterned structure (not shown), which are bonded together via the spacer layer. Thus, the flow channel(s) 12 in the enclosed form of the flow cell 10 is/are defined by the unpatterned structure 15 or patterned structure 17A, 17B, the spacer layer, and either the lid or the second patterned or unpatterned structure.

Alternatively, in the open-wafer form of the flow cell 10, a single unpatterned structure 15 or patterned structure 17A, 17B is included, and the flow channel(s) 12 may be defined by the lane 22 that has been defined in the single patterned structure 17A, 17B or unpatterned structure 15 (e.g., via nanolithography).

The depth of each flow channel 12 in the enclosed versions of the flow cell 10 can be as small as a monolayer thick when microcontact, aerosol, or inkjet printing is used to deposit a separate material (e.g., the spacer layer) that defines at least a portion of the sidewalls of the flow channel 12. This depth could be thicker if the spacer layer is pre-formed or applied via another technique. The depth of the flow channel 12 in some of the open-wafer versions of the flow cell 10 is approximately equivalent to the depth of the lane 22. For other examples, the depth of the flow channel 12 can be about 1 μm, about 10 μm, about 50 μm, about 100 μm, or more. In an example, the depth may range from about 10 μm to about 400 μm. In another example, the depth may range from about 10 μm to about 30 μm. In still another example, the depth is about 5 μm or less. It is to be understood that the depth of the flow channel 12 may be greater than, less than or between the values specified above.

The example flow cell 10 shown in FIG. 1A includes eight flow channels 12. While eight flow channels 12 are shown in FIG. 1A, it is to be understood that any number of flow channels 12 may be included in the flow cell 10 (e.g., a single flow channel 12, four flow channels 12, twelve flow channels 12, etc.). When multiple flow channels 12 are included in the flow cell 10, each flow channel 12 may be isolated from another flow channel 12 so that fluid introduced into one flow channel 12 does not flow into (an) adjacent flow channel(s) 12. In some instances, the spacer layer may be used to fluidly isolate adjacent flow channels 12.

Regardless of the number of flow channels 12 that are included in the flow cell 10, each flow channel 12 may have any desirable shape. In an example, the flow channel 12 has a substantially rectangular configuration with curved ends (as shown in FIG. 1A). The length of the flow channel 12 depends, in part, upon the size of the substrate 14 or 16 used to form the patterned or unpatterned structure 15, 17A, or 17B. The width of each flow channel 12 depends, in part, upon the size of the substrate 14 or 16 used to form the patterned or unpatterned structure 15, 17A, 17B, the desired number of flow channels 12, the desired number of depressions 32 or functionalized pads 36 (when included), and the desired space at a perimeter of the patterned or unpatterned structure 15, 17A, 17B.

Each flow channel 12 that is included in the flow cell 10 may be in fluid communication with an inlet and an outlet (not shown in FIG. 1A through FIG. 1D). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluid(s) to be introduced into the flow channel 12, and the outlet allows fluid(s) to be extracted from the flow channel 12. Each of the inlet(s) and outlet(s) is/are fluidly connected to a fluidic control system (e.g., reservoirs, pumps, valves, waste containers, and the like) that controls fluid introduction and expulsion. Some examples of the fluids that may be introduced into the flow channel(s) 12 include reaction components (e.g., DNA library templates, photodegradable oligonucleotide blockers, polymerases, sequencing primers, nucleotides, etc.), washing solutions, etc.

The flow cell 10 further includes the polymeric hydrogel 30. As described, the flow cell 10 may include the patterned structure 17A shown in FIG. 1C having the plurality of depressions 32 defined in the substrate 14 or 16, and in this example, the polymeric hydrogel 30 is positioned within the depressions 32. As such, some examples of the flow cell 10 include a plurality of depressions 32 defined in the substrate 16, 18, and the polymeric hydrogel 30 is applied within each of the plurality of depressions 32. In these examples, individual depressions 32 (having the polymeric hydrogel 30 therein) are separated from each other individual depression 32 by interstitial regions 34. Alternatively, the flow cell 10 may include the patterned structure 17B shown in FIG. 1D (having the plurality of functionalized pads 36 formed on the substrate 14 or 16). As such, other examples of the flow cell 10 include a plurality of functionalized pads 36 formed on the substrate 14, 16 that are separated by interstitial regions 34, wherein the polymeric hydrogel 30 forms each of the functionalized pads 36. As yet another alternative, the flow cell 10 may include the unpatterned structure 15 shown in FIG. 1B (having the lane 22 defined therein), and in this example, the polymeric hydrogel 30 is positioned within the lane 22. As such, still other examples of the flow cell 10 include the lane 22 defined in the substrate 14, 16, wherein the polymeric hydrogel 30 is positioned within the lane 22.

Many different layouts of the depressions 32 (when included) or functionalized pads 36 (when included) may be envisaged, including regular, repeating, and non-regular patterns. In an example, the depressions 32 or functionalized pads 36 are disposed in a hexagonal grid for close packing and improved density. Other layouts of the depressions 32 or functionalized pads 36 may include, for example, rectilinear (rectangular) layouts, triangular layouts, and so forth. In some examples, the layout or pattern can be an x-y format in rows and columns. In other examples, the layout or pattern can be a repeating arrangement of the depressions 32 or functionalized pads 36 and the interstitial regions 34. In still other examples, the layout or pattern can be a random arrangement of the depressions 32 or functionalized pads 36 (and the interstitial regions 34).

The layout or pattern of the depressions 32 or functionalized pads 36 may be characterized with respect to the density (number) of the depressions 32 or functionalized pads 36 in a defined area. For example, the depressions 32 or functionalized pads 36 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 32 or functionalized pads 36 separated by less than about 100 nm, a medium density array may be characterized as having the depressions 32 or functionalized pads 36 separated by about 400 nm to about 1 μm, and a low density array may be characterized as having the depressions 32 or functionalized pads 36 separated by greater than about 1 μm.

The layout or pattern of the depressions 32 or functionalized pads 36 may also or alternatively be characterized in terms of the average pitch, or the spacing from the center of one depression 32 or functionalized pad 36 to the center of an immediately adjacent depression 32 or functionalized pad 36. Alternatively, the average pitch may refer to the spacing from a left edge of one depression 32 or functionalized pad 36 to the left edge of an immediately adjacent depression 32 or functionalized pad 36. As an additional alternative, average pitch may refer to the spacing from the right edge of one depression 32 or functionalized pad 36 to the right edge of an immediately adjacent depression 32 or functionalized pad 36. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 32 or functionalized pads 36 can be between one of the lower values and one of the upper values selected from the ranges herein.

The size of each of the depressions 32 may be characterized by the volume, opening area, depth, and/or diameter or length and width of the depressions 32. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

The size of each of the functionalized pads 36 may be characterized by the volume or by the length and width of the functionalized pads 36. For example, the volume can range from about 1×10⁻³ μm³ to about 100 μm³, e.g., about 1×10⁻² μm³, about 0.1 μm³, about 1 μm³, about 10 μm³, or more, or less. As another example, the diameter or each of the length and width can range from about 0.1 μm to about 100 μm, e.g., about 0.5 μm, about 1 μm, about 10 μm, or more, or less.

As described and as shown in FIG. 1B through FIG. 1D, the lane 22, the depressions 32, or the functionalized pads 36 include (or are formed using) surface chemistry, where the surface chemistry includes the polymeric hydrogel 30 having at least one primer 26 or 28 attached thereto and a photodegradable oligonucleotide blocker 38 that is hybridized to the at least one primer 26 or 28. As will be described in reference to the methods in FIG. 4 and FIG. 5, the polymeric hydrogel 30 may be pre-grafted or not when it is applied to the substrate 14, 16. Different examples of the pre-grafted polymeric hydrogel are shown at reference numerals 30′, 30″ in FIG. 4.

The polymeric hydrogel 30 may be any gel material that can swell when liquid is taken up and that can contract when liquid is removed, e.g., by drying. In an example, the polymeric hydrogel 30 includes an acrylamide copolymer, such as poly N-(5-azidoacetamidylpentyl)acrylamide-co-acrylamide, PAZAM. PAZAM and some other forms of the acrylamide copolymer are represented by the following structure (I):

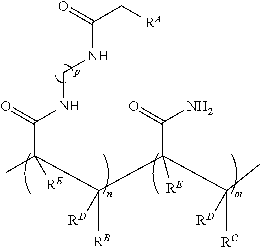

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa. In a specific example, the molecular weight of the acrylamide copolymer is about 312 kDa.

In some examples, PAZAM and other forms of the acrylamide copolymer are linear polymers. In some other examples, PAZAM and other forms of the acrylamide copolymer are lightly cross-linked polymers.

In other examples, the gel material may be a variation of the structure (I). In one example, the acrylamide unit may be replaced with N,N-dimethylacrylamide

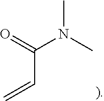

In this example, the acrylamide unit in structure (I) may be replaced with

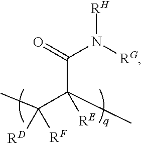

where R^{D}, R^{E} and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

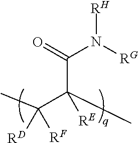

in addition to the recurring “n” and “m” features, where R^{D}, R^{E} and R^{F} are each H or a C1-C6 alkyl, and R^{G} and R^{H} are each a C1-C6 alkyl. In this example, q may be an integer in the range of 1 to 100,000.

As another example of the polymeric hydrogel 30, the recurring “n” feature in structure (I) may be replaced with a monomer including a heterocyclic azido group having structure (II):

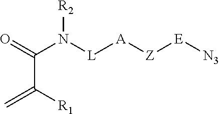

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymeric hydrogel 30 may include a recurring unit of each of structure (III) and (IV):

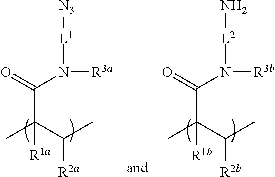

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each of L¹ and L² is independently selected from an optionally substituted alkylene linker or an optionally substituted heteroalkylene linker.

It is to be understood that other polymeric hydrogels 30 may be used, provided that the hydrogels are suitable for grafting oligonucleotide primers 26, 28 thereto. Some additional examples of suitable materials for the polymeric hydrogel 30 include functionalized polysilanes, such as norbornene silane, azido silane, alkyne functionalized silane, amine functionalized silane, maleimide silane, or any other polysilane having functional groups that can attach the desired primer set 26, 28. Other examples of suitable polymeric hydrogels 30 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2]photo-cycloaddition reactions. Still other examples of suitable polymeric hydrogels include mixed copolymers of acrylamides and acrylates. A variety of polymer architectures containing acrylic monomers (e.g., acrylamides, acrylates etc.) may be utilized in the examples disclosed herein, such as branched polymers, including dendrimers, and the like. For example, the monomers (e.g., acrylamide, etc.) may be incorporated, either randomly or in block, into the branches (arms) of a dendrimer.

The polymeric hydrogel 30 may be formed using any suitable copolymerization process and may be deposited using any of the methods disclosed herein. For at least some of the deposition techniques, the polymeric hydrogel 30 may be incorporated into a mixture, e.g., with water or with ethanol and water, and then applied within the lane 22, within the depression(s) 32, or to form the functionalized pad(s) 36. In some instances, the polymeric hydrogel 30 is a cured hydrogel (e.g., that has been cured using heat, UV/high energy light, or the like).

The attachment of the polymeric hydrogel 30 to the substrate 14 or to the layer 20 of the multi-layer substrate 16 may be through covalent bonding. As will be described in more detail in regard to the methods depicted in FIG. 4 and FIG. 5, in some instances, the substrate 14 or the layer 20 may be activated before the polymeric hydrogel 30 is applied thereon, e.g., through silanization or plasma ashing. Activation of the substrate 14 or layer 20, when performed, facilitates the attachment of the polymeric hydrogel 30 to the substrate 14 or layer 20. Covalent linking is helpful for maintaining the primers 26, 28 at desired regions of the substrate 14 or layer 20 throughout the lifetime of the flow cell 10 and during a variety of uses.

As described and as shown in FIG. 1B through FIG. 1D, the polymeric hydrogel 30 includes a plurality of primers 26, 28 attached thereto, where each of the plurality of primers 26, 28 is respectively hybridized to a photodegradable oligonucleotide blocker 38. These primers 26, 28 may form a primer set.

In an example, the primers 26, 28 may be amplification primers. In this example, the amplification primers 26, 28 can be immobilized to the polymeric hydrogel 30 by single point covalent attachment at or near the 5′ end of the primers 26, 28. This attachment leaves i) an adapter-specific portion of the primers 26, 28 free to anneal to its cognate sequencing-ready nucleic acid fragment and ii) the 3′ hydroxyl group free for primer extension (after the hybridized photodegradable oligonucleotide blocker 38 has been removed from the primer 26, 28, as will be described herein). Any suitable covalent attachment may be used for this purpose. Examples of terminated primers that may be used include alkyne terminated primers (e.g., which may attach to an azide surface moiety of the polymeric hydrogel 30), or azide terminated primers (e.g., which may attach to an alkyne surface moiety of the polymeric hydrogel 30), or phospho-thioate terminated primers (e.g., which may attach to bromine surface moieties of the polymeric hydrogel 30).

The two different primers 26, 28 of the primer set may be used in sequential paired end sequencing. Together, the primers 26, 28 of the set enable amplification of library templates that include corresponding adapters at opposed ends. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC or PD primer, or any combination of one PC primer and one PD primer.

Specific examples of suitable primers 26, 28 include P5 and P7 primers used on the surface of commercial flow cells sold by Illumina Inc. for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms.

The P5 primer (shown as a cleavable primer due to the cleavable nucleobase uracil or “n”) is:

P5 #1: 5′→3′
(SEQ. ID. NO. 1)
AATGATACGGCGACCACCGAGAUCTACAC;
or
P5 #2: 5′→3′
(SEQ. ID. NO. 2)
AATGATACGGCGACCACCGAGAnCTACAC

where “n” is alkene-thymidine (i.e., alkene-dT) in SEQ. ID. NO. 2; or

P5 #3: 5′→3′
(SEQ. ID. NO. 3)
AATGATACGGCGACCACCGAGAnCTACAC

where “n” is inosine in SEQ. ID. NO. 3.

The P7 primer (shown as cleavable primers) may be any of the following:

P7 #1: 5′→3′
(SEQ. ID. NO. 4)
CAAGCAGAAGACGGCATACGAnAT

where “n” is 8-oxoguanine;

P7 #2: 5′→3′
(SEQ. ID. NO. 5)
CAAGCAGAAGACGGCATACnAGAT

where “n” is 8-oxoguanine;

P7 #3: 5′→3′
(SEQ. ID. NO. 6)
CAAGCAGAAGACGGCATACnAnAT

where each instance of “n” is 8-oxoguanine.

The P15 primer (shown as a cleavable primer) is:

P15: 5′→3′
(SEQ. ID. NO. 7)
AATGATACGGCGACCACCGAGAnCTACAC

where “n” is allyl-T (a thymine nucleotide analog having an allyl functionality).

The other primers (PA-PD, shown as non-cleavable primers) mentioned above include:

PA 5′→3′
(SEQ. ID. NO. 8)
GCTGGCACGTCCGAACGCTTCGTTAATCCGTTGAG
PB 5′→3′
(SEQ. ID. NO. 9)
CGTCGTCTGCCATGGCGCTTCGGTGGATATGAACT
PC 5′→3′
(SEQ. ID. NO. 10)
ACGGCCGCTAATATCAACGCGTCGAATCCGCAACT
PD 5′→3′
(SEQ. ID. NO. 11)
GCCGCGTTACGTTAGCCGGACTATTCGATGCAGC

While not shown in the example sequences for PA-PD, it is to be understood that any of these primers may include a cleavage site, such as uracil, 8-oxoguanine, allyl-T, etc. at any point in the strand. When two primers 26, 28 are used in a primer set, it is to be understood that the cleavage sites of the two primers are orthogonal. In this regard, “orthogonal” means that the cleavage site of one primer 26 in the set is not susceptible to the cleaving agent used for the cleavage site of the other primer 28 in the set. Thus, the cleavage of one cleavage site will not affect the other cleavage site. Thus, while example cleavage sites are provided for the primer sequences, it is to be understood that each primer sequence may include a different cleavage site (e.g., P7 could include U instead of 8-oxoguanine, as long as the primer is paired, in the set, with a primer having an orthogonal cleavage site.

Each of the primers 26, 28 disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer 26, 28 may also include a linker. Any linker that includes a terminal alkyne group or another suitable terminal functional group that can attach to the surface functional groups of the polymeric hydrogel 30 may be used, and thus, the terminal functional group selected will depend, in part, upon the functional groups included in the polymeric hydrogel 30. As such, in some examples, each of the plurality of primers 26, 28 includes a linker, and each of the plurality of primers 26, 28 is respectively attached to the polymeric hydrogel 30 via the linker. In one example, the linkers of the primers 26, 28 include terminal hexynyl functional groups or internal alkynes (e.g., bicyclononyne).

FIG. 1B, FIG. 1C, and FIG. 1D further depict a plurality of photodegradable oligonucleotide blockers 38 respectively hybridized to at least some of the plurality of primers 26, 28 attached to the polymeric hydrogel 30. Each of the plurality of photodegradable oligonucleotide blockers 38 shown in FIG. 1B through FIG. 1D includes a nucleotide-based backbone having a photodegradable moiety 40 incorporated therein.

The nucleotide-based backbone of each of the photodegradable oligonucleotide blockers 38 includes a chain of linked nucleotides (e.g., adenine, guanine, cytosine, thymine, uracil, etc.) with individual units of a photodegradable moiety 40 interspersed throughout the chain of linked nucleotides. The individual nucleotides included in the nucleotide-based backbone are selected to be complementary to at least some of the nucleotides included in the primers 26, 28, such that the nucleotides in the backbone of each oligonucleotide blocker 38 and at least some of the nucleotides making up each of the primers 26, 28 can hybridize to one another (e.g., via base-pair or hydrogen bond interactions). In some instances, the nucleotides included in the nucleotide-based backbone of the blockers 38 are selected to be complementary to specific nucleotides within the primers 26, 28, such that the hybridization of the blockers 38 to the primers 26, 28 creates an overhang of each blocker 38 on its respective primer 26, 28 at the 3′ end of the primer 26, 28. In one example, the overhang can be achieved by utilizing a blocker 38 that is longer than the primer 26, 28 to which it is to hybridize. In another example, the overhang can be achieved by utilizing a blocker 38 that is mismatched at and near the 5′ end of the primer 26, 18 and complementary at and near the 3′ end of the primer 26, 28. The overhang configuration may be suitable for techniques in which strand displacement is to occur during blocker 38 removal. In any instance, the nucleotides included in the nucleotide-based backbone of the blockers 38 are selected to be i) complementary to a sufficient number of the primer nucleotides and ii) positioned such that the 3′ end is hybridized and the kinetics favor hybridization over dissociation.

Individual units of the photodegradable moiety 40 may be interspersed throughout the nucleotide-based backbone of the blockers 38 in any desired configuration disclosed herein. As examples, the individual units of the photodegradable moiety 40 may be evenly spaced throughout the nucleotide-based backbone of the blockers 38, or concentrated at specific areas of the nucleotide-based backbone, or indiscriminately interspersed throughout the nucleotide-based backbone. In a specific example, the units of the photodegradable moiety 40 are concentrated within the blocker 38 at a specific area of the blocker 38 that hybridizes near the 5′ end of the primers 26, 28 or near the 3′ end of the primers 26, 28. In this example, the degradation of the photodegradable moiety 40 (during use or preparation of the flow cell 10) creates a “toehold,” or a small unblocked section of the primer 26, 28, that can seed a portion of a (DNA) library template thereto, while the remainder of the blocker 38 remains bound to the primer 26, 28. When the portion of the DNA library template becomes seeded to the toehold during use or preparation of the flow cell 10, the enzyme free removal of the remaining portion of the oligonucleotide blocker 38 from the primer 26, 28 (by the competing DNA library template) becomes thermodynamically favored. In effect, the incoming DNA library template partially seeds and drives off the remainder of the blocker 38.

In examples, the photodegradable moiety 40 incorporated into the nucleotide-based backbone of the blocker 38 is an ortho-nitrobenzyl containing moiety, as this term is defined herein. Examples of the ortho-nitrobenzyl containing moiety have the following structure:

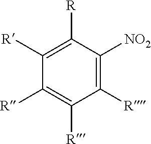

where each of R, R′, R″, R″′, and R″″ is independently selected from the group consisting of a hydrogen, a halogen, an alcohol, an ether, an ester, and a linear or branched alkyl including a terminal carboxyl group, a terminal amino group, or a terminal phosphate group. Thus, the R groups (e.g., R, R′, R″, R″′, R″″) may be any combination of the listed functional groups. Other example ortho-nitrobenzyl containing moieties are multi-ringed structures that include the NO₂ substituted phenyl ring.

In some specific examples, the ortho-nitrobenzyl containing moiety is selected from the group consisting of

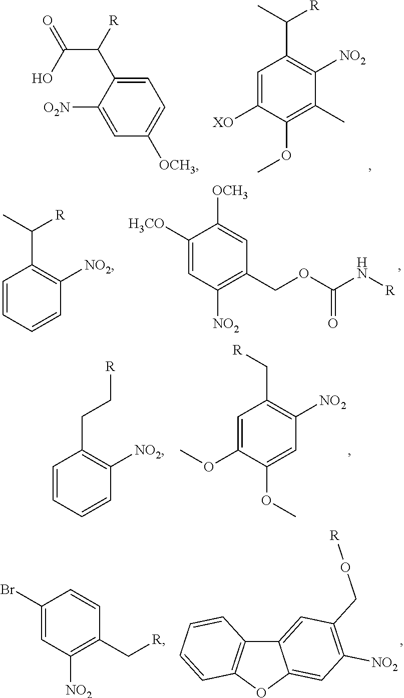

and a combination thereof, where in each of the structures: X is any halogen group, and R is one of a carboxyl group, an amino group, or a phosphate group.

It is to be understood that other photodegradable moieties 40 may be used that are responsive to suitable preselected light wavelengths and that may be incorporated into the nucleotide-based backbone of the oligonucleotide blocker(s) 38. It is to be further understood that individual units of different examples (i.e., different chemical structures) of the photodegradable moiety 40 may be incorporated into a single nucleotide-based backbone, or individual units of the same type of the photodegradable moiety 40 may be incorporated into a single nucleotide-based backbone.

The photodegradable moiety 40 (or moieties 40) incorporated into the nucleotide-based backbone of the blocker 38 are selected to be responsive (e.g., susceptible to degradation) at a preselected range of light wavelengths. In examples, the photodegradable moiety 40 is susceptible to ultraviolet (UV) light (e.g., at light wavelengths ranging from about 100 nm to about 400 nm). In a specific example, the photodegradable moiety 40 incorporated into the nucleotide-based backbone is photodegradable at a light wavelength ranging from about 300 nm to about 450 nm. The light wavelength that is used will depend, in part, upon the chemical structure(s) of the photodegradable moiety/moieties 40 that is/are included in the nucleotide-based backbone. Exposure of the photodegradable moiety 40 to the preselected wavelength of light results in a chemical transformation of the photodegradable moiety 40 that either removes the oligonucleotide blocker 38 from the primers 26, 28, or that renders the oligonucleotide blocker 38 (including the moiety 40) susceptible to conditions that facilitate the removal of the oligonucleotide blocker 38.

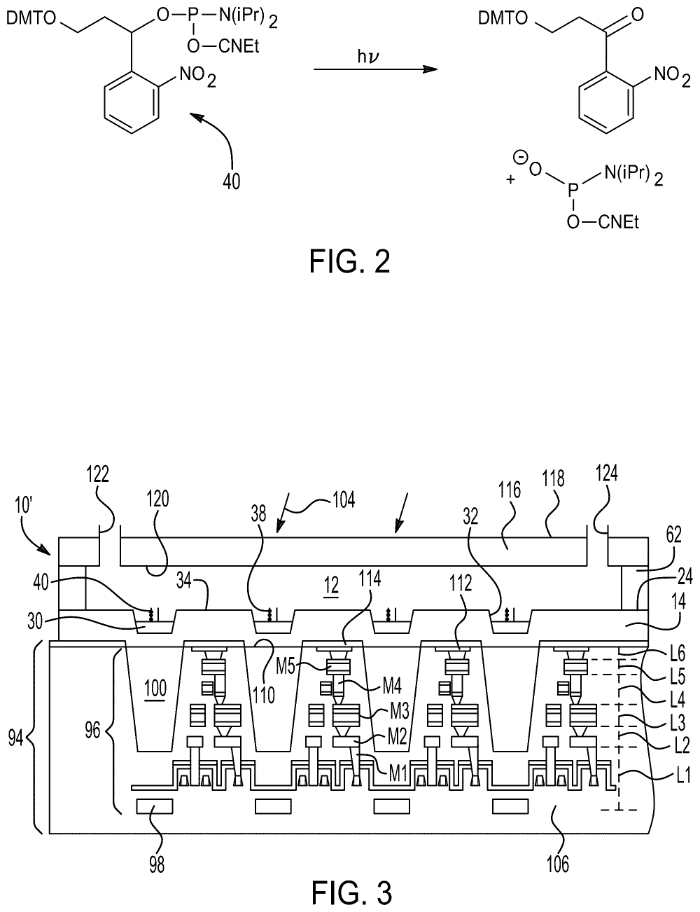

A specific example of a suitable chemical structure that may be used for the photodegradable moiety 40 is shown on the left in FIG. 2. The chemical structure shown on the left in FIG. 2 is an ortho-nitrobenzyl phosphoramidite structure, where DMT is dimethoxytrityl and protects the 5′ hydroxyl group on deoxyribose. The structure shown on the left has not been exposed to light and the dehybridization of the nucleotide-based backbone of the blocker 38 from the primers 26, 28 is kinetically disfavored (i.e., the rate of dissociation of the blocker(s) 38 from the primer(s) 26, 28 is low). FIG. 2 further depicts a chemical transformation undergone by the photodegradable moiety 40 upon exposure to a preselected wavelength of light. The structure resulting from the chemical transformation is shown on the right in FIG. 2. As shown, when the structure on the left is exposed to a preselected wavelength of light (e.g., light ranging from 300 nm to 450 nm, etc.), the phosphoramidite becomes excised from the moiety 40, and the resulting chemical structure will readily dehybridize from the primers 26, 28 in a kinetically/thermodynamically favorable manner (i.e., the rate of dissociation of the blocker(s) 38 from the primer(s) 26, 28 substantially increases after the transformation).

The rate of dissociation of the blocker(s) 38 from the primer(s) 26, 28 prior to exposure of the primers 26, 28 to the preselected wavelength of light is low enough that the dissociation is kinetically disfavored (until the blockers 38 are exposed to the preselected wavelength of light). The particular rate of dissociation of the blockers 38 from the primers 26, 28 prior to light exposure will vary based, in part, upon the number of individual nucleotides and the number of individual units of the photodegradable moiety 40 included in the nucleotide-based backbone of each blocker 38. Generally, the inclusion of more nucleotides in the nucleotide-based backbone will decrease the rate of dissociation prior to light exposure, and the inclusion of fewer nucleotides in the nucleotide-based backbone will increase the rate of dissociation prior to light exposure.

The number of individual nucleotides and/or individual units of the photodegradable moiety 40 included in the nucleotide-based backbone of the oligonucleotide blocker(s) 38 may be expressed numerically. In some numerically-expressed examples, the nucleotide-based backbone includes from 10 nucleotides to 250 nucleotides, or from 10 nucleotides to 150 nucleotides, or from 10 nucleotides to 100 nucleotides, or from 10 nucleotides to 50 nucleotides, etc. In these numerically-expressed examples, the number of individual units of the photodegradable moiety 40 incorporated into the nucleotide-based backbone ranges from 2 to 10. In further examples, the number of individual units of the photodegradable moiety 40 incorporated into the nucleotide-based backbone ranges from 2 to 5, or from 2 to 20. In a specific numerically-expressed example, the nucleotide-based backbone includes from 10 nucleotides to 150 nucleotides, and the number of individual units of the photodegradable moiety 40 incorporated into the nucleotide-based backbone ranges from 2 to 10.

Alternatively, the number of individual nucleotides and/or individual units of the photodegradable moiety 40 incorporated into the nucleotide-based backbone of the oligonucleotide blocker(s) 38 may be expressed as a percentage. In an example, a percentage of the photodegradable moiety 40 incorporated into the nucleotide-based backbone ranges from about 0.01% to about 30%, relative to a total number of individual units of the photodegradable moiety 40 plus a total number of individual nucleotides included in the nucleotide-based backbone of the blocker(s) 38.

Regardless of the number of individual units of the photodegradable moiety 40 that are included therein, the photodegradable oligonucleotide blocker 38 can be prepared using standard DNA synthesis techniques (e.g., using a phosphoramidite addition process, which in simplified form involves de-blocking, coupling, capping, and oxidation within each base addition cycle).

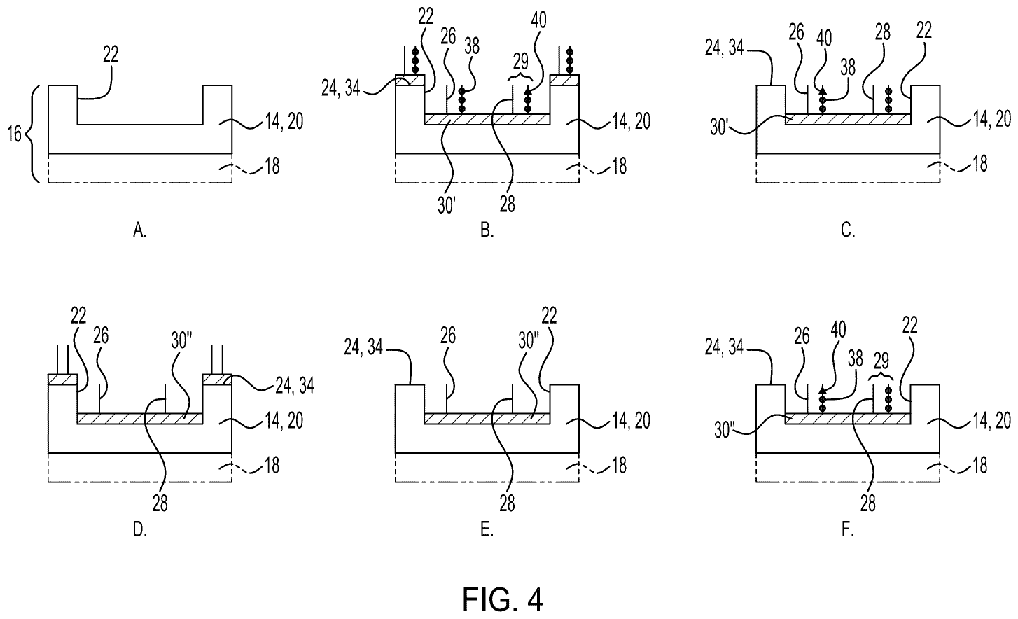

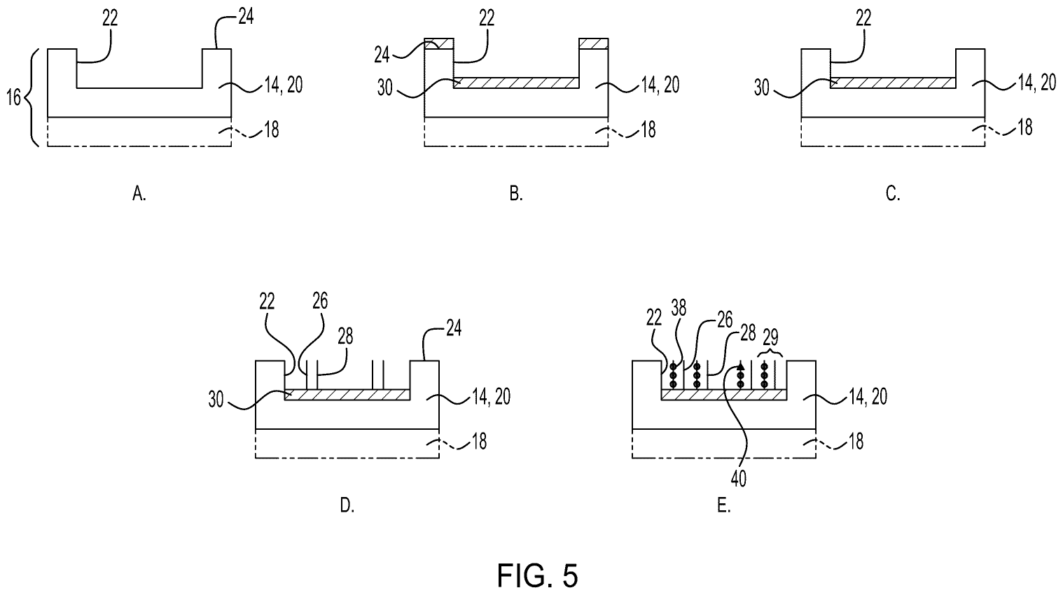

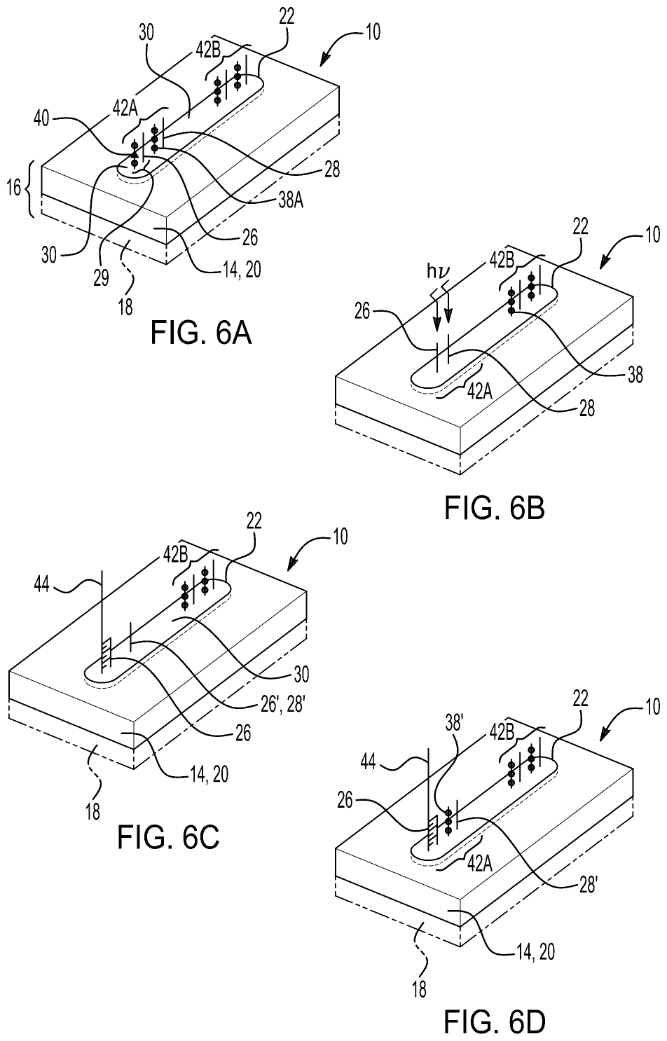

In some instances, the flow cell 10 further includes a complementary metal oxide semiconductor (CMOS) chip 94 coupled to a bottom of the substrate 14, which forms the flow cell 10′ shown in FIG. 3.

While the flow cell 10′ depicted in FIG. 3 is shown as an enclosed version with a lid 116, it is to be understood that other enclosed versions of the flow cell 10′ may be used, such as a flow cell 10′ including two patterned structures 17A, 17B that are bonded together. Further, open-wafer versions of the flow cell 10′ may be used, where a single patterned structure 17A, 17B is open to the surrounding environment and is coupled to the CMOS chip 94.

As described, in addition to the complementary metal oxide semiconductor chip 94, this example flow cell 10′ includes the substrate 14 over the complementary metal oxide semiconductor chip 94. For ease of illustration, the substrate 14 is shown in FIG. 3. It is to be understood, however, that the multi-layer substrate 16 could be used instead, where the CMOS chip 94 is coupled to the substrate 16 via attachment to the base support 18. For further ease of illustration, the substrate 14 of the flow cell 10′ of FIG. 3 is shown as including a plurality of depressions 32 separated by interstitial regions 34. While the flow cell 10′ shown in FIG. 3 includes the depressions 32 (similar to the patterned structure 17A shown in FIG. 1C), the flow cell 10′ of FIG. 3 may include a plurality of functionalized pads 36 separated by interstitial regions 34, where each functionalized pad 36 includes the polymeric hydrogel 30, the primers 26, 28, and the photodegradable oligonucleotide blockers 38 (similar to the patterned structure 17B shown in FIG. 1D). As an additional alternative, it is to be understood that the substrate 14, 16 of the flow cell 10′ may alternatively include the lane 22 defined therein, where the lane 22 has the polymeric hydrogel 30, the primers 26, 28, and the photodegradable oligonucleotide blockers 38 therein (similar to the unpatterned structure 15 shown in FIG. 1B).

In the illustrated example, the substrate 14 of the flow cell 10′ may be affixed directly to, and thus be in physical contact with, the CMOS chip 94 through one or more securing mechanisms (e.g., adhesive, bond, fasteners, and the like). It is to be understood that the substrate 14 may be removably coupled to the CMOS chip 94.

The CMOS chip 94 includes a plurality of stacked layers 96 including, for example, silicon layer(s), dielectric layer(s), metal-dielectric layer(s), metal layer(s), etc.). The stacked layers 96 make up the device circuitry, which includes detection circuitry.

The CMOS chip 94 includes optical components, such as optical sensor(s) 98 and optical waveguide(s) 100. The optical components may be arranged such that each optical sensor 98 at least substantially aligns with, and thus is operatively associated with, a single optical waveguide 100 and a single depression 32 (or functionalized pad 36) of the flow cell 10′. However, in other examples, a single optical sensor 98 may receive photons through more than one optical waveguide 100 and/or from more than one depression 32 (or functionalized pad 36). In these other examples, the single optical sensor 98 is operatively associated with more than one optical waveguide 100 and/or more than one depression 32 (or functionalized pad 36).

As used herein, a single optical sensor 98 may be a light sensor that includes one pixel or more than one pixel. As an example, each optical sensor 98 may have a detection area that is less than about 50 μm². As another example, the detection area may be less than about 10 μm². As still another example, the detection area may be less than about 2 μm². In the latter example, the optical sensor 98 may constitute a single pixel. An average read noise of each pixel of the optical sensor 98 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 100 may be a light guide including a cured filter material that i) filters the excitation light 104 (propagating from an exterior of the flow cell 10′ into the flow channel 12), and ii) permits the light emissions resulting from reactions at the depressions 32 or functionalized pads 36 (not shown) to propagate therethrough toward corresponding optical sensor(s) 98. In an example, the optical waveguide 100 may be, for example, an organic absorption filter. As a specific example, the organic absorption filter may filter excitation light 104 of about 532 nm wavelength and permit light emissions of about 570 nm or more wavelengths. The optical waveguide 100 may be formed by first forming a guide cavity in a dielectric layer 106, and then filling the guide cavity with a suitable filter material.

The optical waveguide 100 may be configured relative to the dielectric material 106 in order to form a light-guiding structure. For example, the optical waveguide 100 may have a refractive index of about 2.0 so that the light emissions are substantially reflected at an interface between the optical waveguide 100 and the surrounding dielectric material 106. In certain examples, the optical waveguide 100 is selected such that the optical density (OD) or absorbance of the excitation light 104 is at least about 4 OD. More specifically, the filter material may be selected and the optical waveguide 100 may be dimensioned to achieve at least 4 OD. In other examples, the optical waveguide 100 may be configured to achieve at least about 5 OD or at least about 6 OD.

The substrate 14 (or the base support 18 of the substrate 16) functions as a passivation layer for the flow cell 10′. At least a portion of the substrate 14 is in contact with a first embedded metal layer 112 of the CMOS chip 94 and also with an input region 110 of the optical waveguide 100. The contact between the substrate 14 and the first embedded metal layer 112 may be direct contact or may be indirect contact through a shield layer 114.

The substrate 14 (passivation layer) may provide one level of corrosion protection for the embedded metal layer 112 of the CMOS chip 94 that is closest in proximity to the substrate 14. In this example, the substrate 14 may include a passivation material that is transparent to the light emissions resulting from reactions within the depressions 32 (e.g., visible light), and that is at least initially resistant to the fluidic environment and moisture that may be introduced into or present in the flow channel 12. An at least initially resistant material acts as an etch barrier to high pH reagents (e.g., pH ranging from 8 to 14) and as a moisture barrier. Examples of suitable materials for the substrate 14 of the flow cell 10′ include silicon nitride (Si₃N₄), silicon oxide (SiO₂), tantalum pentoxide (Ta₂O₅), hafnium oxide (HfO₂), boron doped p+silicon, or the like. The thickness of the substrate 14 may vary depending, in part upon the sensor dimensions. In an example, the thickness of the substrate 14 ranges from about 100 nm to about 500 nm.

As described, in the example shown in FIG. 3, the flow cell 10′ also includes a lid 116 that is operatively connected to the substrate 14 to partially define the flow channel 12 between the substrate 14 (and the depressions 32 or pads 36 therein) and the lid 116. The lid 116 may be any material that is transparent to the excitation light 104 that is directed toward the depressions 32 (or toward the pads 36). As examples, the lid 116 may include glass (e.g., borosilicate, fused silica, etc.), plastic, etc. A commercially available example of a suitable borosilicate glass is D 263®, available from Schott North America Inc. Commercially available examples of suitable plastic materials, namely cyclo olefin polymers, are the ZEONOR® products available from Zeon Chemicals L.P.

The lid 116 may be physically connected to the substrate 14 through material 62. In the example shown in FIG. 3, the material 62 is/are coupled to a portion the surface of the substrate 14 (e.g., at bonding regions 24 of the substrate 14). The material 62 also extends between the surface of the substrate 14 and an interior surface of the lid 116. In some examples, the material 62 and the lid 116 may be integrally formed such that they 62, 116 are a continuous piece of material (e.g., glass or plastic). In these examples, a thin layer of adhesive may be used to attach the integrally formed piece to the substrate 14 at the bonding region 24. In other examples, the material 62 and the lid 116 may be separate components that are coupled to each other. In these other examples, the material 62 may be the same material as, or a different material than the lid 116. In still other examples, the material 62 includes a curable adhesive layer that bonds the lid 116 to the substrate 14 (at a portion of its surface). This material 62 is similar to the spacer layer described herein.

In an example, the lid 116 may be a substantially rectangular block having an at least substantially planar exterior surface 118, and an at least substantially planar interior surface 120 that defines a portion of the flow channel 12. The block may be mounted onto the material 62. Alternatively, the block may be etched to define the lid 116 and the material 62 (which functions as sidewall(s)). For example, a recess may be etched into the transparent block. When the etched block is mounted to the substrate 14, the recess may become the flow channel 12.

The lid 116 may include inlet and outlet ports 122, 124 that are configured to fluidically engage other ports (not shown) for directing fluid(s) into the flow channel 12 (e.g., from a reagent cartridge or other fluid storage system component) and out of the flow channel 12 (e.g., to a waste removal system).

The flow channel 12 may be sized and shaped to direct a fluid along the depressions 32, along the lane 22, or over the functionalized pads 36, depending on the unpatterned or patterned structure 15, 17A, or 17B that is used in the flow cell 10′. The height of the flow channel 12 and other dimensions of the flow channel 12 may be configured to maintain a substantially even flow of the fluid over the depressions 32, the lane 22, or the functionalized pads 36. The dimensions of the flow channel 12 may also be configured to control bubble formation. In an example, the height of the flow channel 12 may range from about 50 μm to about 400 μm. In another example, the height of the flow channel 12 may range from about 80 μm to about 200 μm. It is to be understood that the height of the flow channel 12 may vary.

Each depression 32 or functionalized pad 36, when included in the flow cell 10′, is a localized region in the substrate 14 of the flow cell 10′ where a designated reaction may occur. In an example, each depression 32 (or functionalized pad 36) is at least substantially aligned with the input region 110 of a single optical waveguide 100. As such, light emissions at the depressions 32 or functionalized pads 36 may be directed into the input region 110, through the waveguide 100, and to an associated optical sensor 98. In other examples, one depression 32 or functionalized pad 36 may be aligned with several input regions 110 of several optical waveguides 100. In still other examples, several depressions 32 or functionalized pads 36 may be aligned with one input region 110 of one optical waveguide 100.

The embedded metal layer 112 may be any suitable CMOS metal, such as aluminum (Al), aluminum chloride (AICI), tungsten (W), nickel (Ni), or copper (Cu). In an example, the embedded metal layer 112 may be a functioning part of the CMOS AVdd line, and through the stacked layers 96, is also electrically connected to the optical sensor 98. Thus, the embedded metal layer 112 participates in the detection/sensing operation.

It is to be understood that the other optical sensors 98 and associated components may be configured in an identical or similar manner. It is also to be understood, however, that the CMOS chip 94 may not be manufactured identically or uniformly throughout. Instead, one or more optical sensor 98 and/or associated components may be manufactured differently or have different relationships with respect to one another.

The stacked layer 96 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 94.

The CMOS chip 94 may be manufactured using integrated circuit manufacturing processes. The CMOS chip 94 may include multiple layers, such as a sensor base/layer (e.g., a silicon layer or wafer). The sensor base may include the optical sensor 98. When the CMOS chip 94 is fully formed, the optical sensor 98 may be electrically coupled to the rest of the circuitry in the stack layer 96 through gate(s), transistor(s), etc.

As used in reference to FIG. 3, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layer 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 106 (e.g., SiO₂). Various metallic elements M1-M5 and dielectric materials 106 may be used, such as those suitable for integrated circuit manufacturing.

In the example shown in FIG. 3, each of the plurality of metal-dielectric layers L1-L6 includes both metallic elements M1, M2, M3, M4, M5 and the dielectric material 106. In each of the layers L1-L6, the metallic elements M1, M2, M3, M4, M5 are interconnected and are embedded within dielectric material 106. In some of the metal-dielectric layers L1-L6, additional metallic elements may also be included. Some of these additional metallic elements may be used to address individual pixels through a row and column selector. The voltages at these elements may vary and switch between about −1.4 V and about 4.4 V depending upon which pixel the device is reading out.

The configuration of the metallic elements M1, M2, M3, M4, M5 and the dielectric layer 106 in FIG. 3 is illustrative of the circuitry, and it is to be understood that other examples may include fewer or additional layers and/or may have different configurations of the metallic elements M1-M5.

In the example shown in FIG. 3, the shield layer 114 is in contact with at least a portion of the substrate 14. The shield layer 114 has an aperture at least partially adjacent to the input region 110 of the optical waveguide 100. This aperture enables the depressions 32 or pads 36 (and at least some of the light emissions therefrom) to be optically connected to the waveguide 100. It is to be understood that the shield layer 114 may have an aperture at least partially adjacent to the input region 110 of each optical waveguide 100. The shield layer 114 may extend continuously between adjacent apertures.

The shield layer 114 may include any material that can block, reflect, and/or significantly attenuate the light signals that are propagating through the flow channel 12. The light signals may be the excitation light 104 and/or the light emissions from the depressions 32. As an example, the shield layer 114 may be tungsten (W).

It is to be understood that the flow cell 10′ may also be used for optical detection.

### Methods Utilizing a Pre-Grafted Polymeric Hydrogel

Different methods of applying the polymeric hydrogel 30, of attaching the primers 26, 28 to the polymeric hydrogel 30, and of hybridizing the photodegradable oligonucleotide blockers 38 to the primers 26, 28 (e.g., to prepare flow cells 10, 10′) will now be described. These methods generally include applying the polymeric hydrogel 30 over at least a portion of a surface of a substrate 14, 16; attaching a plurality of primers 26, 28 to the polymeric hydrogel 30; and hybridizing a plurality of photodegradable oligonucleotide blockers 38 to at least some of the plurality of primers 26, 28.

Two methods that may be used as part of a process of generating the flow cell 10 or 10′ are respectively shown in FIG. 4 at A., B., C. and in FIG. 4 at A., D., E., F. In either of the two methods shown in FIG. 4, the polymeric hydrogel 30 that is used is a pre-grafted polymeric hydrogel 30′ or 30″, meaning that at least the primers 26, 28 are/become attached to the polymeric hydrogel 30 before the hydrogel 30′ or 30″ is deposited over the substrate 14, 16. As such, in either of the two methods shown in FIG. 4, the attaching of the plurality of primers 26, 28 (with or without the photodegradable oligonucleotide blockers 38 hybridized thereto) to the polymeric hydrogel 30 is performed prior to applying the polymeric hydrogel 30′ or 30″ over the at least the portion of the surface of the substrate. The primers 26, 28 (with or without the photodegradable oligonucleotide blockers 38 hybridized thereto) may be pre-grafted to the hydrogel 30 (to form the pre-grafted polymeric hydrogel 30′ or 30″) using any suitable grafting technique. As an example, a primer solution or mixture may be formed, where the solution or mixture includes the primer(s) 26, 28, the polymeric hydrogel 30, water, a buffer, and a catalyst. Functional groups at or near the 5′ end of the primers 26, 28 in the solution or mixture react with reactive surface functional groups of the polymeric hydrogel 30 and become attached thereto, thereby forming the pre-grafted polymeric hydrogel 30′ or 30″.

Both example methods shown in FIG. 4 begin at A. As shown in FIG. 4 at A., the substrate 14 (or the layer 20 of the substrate 16) has a lane 22 defined therein (similar to the unpatterned structure 15 depicted in FIG. 1B). It is to be understood, however, that the example methods shown in FIG. 4 may alternatively utilize a substrate 14, 16 having depressions 32 defined therein (similar to the patterned structure 17A in FIG. 1C). In any case, either the single layer substrate 14 or the multi-layer substrate 16 including the base support 18 and the layer 20 may be used in these example methods.

The lane 22 may be defined in the substrate 14 (or in the layer 20 of the substrate 16) using any suitable technique described herein (e.g., etching, nanoimprint lithography, photolithography, etc.). The material of the substrate 14 or the material of the components of the substrate 16 (i.e., the material of the base support 18 and the layer 20) may be any suitable example set forth herein. The patterning technique that is used for the substrate 14 or the layer 20 will depend, in part, upon the material selected for the substrate 14 or the layer 20.

As an example of forming the lane 22, when the substrate 14 or the layer 20 includes a resin material, a working stamp (including a negative replica of the lane 22) may be pressed into the resin material of the substrate 14 or layer 20 while the resin is soft. The resin of the substrate 14 or layer 20 may then be cured while the working stamp is in place, e.g., via exposure to actinic radiation or to heat. After curing, the working stamp is released, which forms the lane 22 in the substrate 14 or in the layer 20. As an example of forming the depressions 32 (when included), when the substrate 14 or the layer 20 includes a resin material, a working stamp (including a negative replica of the depressions 32) may be pressed into the resin material of the substrate 14 or layer 20 while the resin is soft. The resin of the substrate 14 or layer 20 may then be cured while the working stamp is in place, e.g., via exposure to actinic radiation or to heat. After curing, the working stamp is released, which forms the depressions 32 in the substrate 14 or in the layer 20.

One of the methods shown in FIG. 4 proceeds from A. to B. In examples of this method, a “pre-hybridized” form of the pre-grafted polymeric hydrogel 30′ is applied over the substrate 14, 16. In these examples, the pre-grafted polymeric hydrogel 30′ is referred to as being “pre-hybridized” because the oligonucleotide blockers 38 are individually hybridized to respective primers 26, 28 (that are attached to the pre-grafted polymeric hydrogel 30′) before the pre-grafted polymeric hydrogel 30′ is applied over the substrate 14, 16. The pre-hybridization of the primers 26, 28 to the oligonucleotide blockers 38 forms a plurality of double-stranded entities 29 on the pre-grafted polymeric hydrogel 30′. As such, in examples of the method that proceed from A. to B. in FIG. 4, prior to attaching the plurality of primers 26, 28 to the polymeric hydrogel 30, the photodegradable oligonucleotide blockers 38 are respectively hybridized to the at least some of the plurality of primers 26, 28 to form a plurality of double stranded entities 29; and attaching the plurality of primers 26, 28 to the polymeric hydrogel 30 involves attaching the plurality of double stranded entities 29 to the polymeric hydrogel 30. The plurality of double-stranded entities 29 may be formed by incubating the primers 26, 28 and the oligonucleotide blockers 38 in a suitable reaction vessel, whereby the nucleotides in the blockers 38 and the nucleotides in the primers 26, 28 hybridize to one another during the incubation process. The double-stranded entities 29 may then be attached to the polymeric hydrogel 30 through the 5′ end of the primers 26, 28 and the R^{A} groups of the polymeric hydrogel 30 (e.g., via copper mediated or copper free click reactions depending upon the functional groups). This forms the pre-grafted, pre-hybridized polymeric hydrogel 30′.

The primers 26, 28 used in this example method may include any example of the P5, P7, P15, and PA-PD primers 26, 28 disclosed herein. In examples, the primers 26, 28 form a set, where each primer 26, 28 is respectively complementary to a designated adapter sequence on DNA library templates that are to be seeded to the primers 26, 28.

In this example method, each of the plurality of photodegradable oligonucleotide blockers 38 includes the nucleotide-based backbone having the photodegradable moiety 40 incorporated therein. The photodegradable moiety 40 (or moieties 40) may be any suitable example(s) described herein. In some examples, the photodegradable moiety 40 incorporated into the nucleotide-based backbone is an ortho-nitrobenzyl containing moiety. In some of these examples, the ortho-nitrobenzyl containing moiety is selected from the group consisting of:

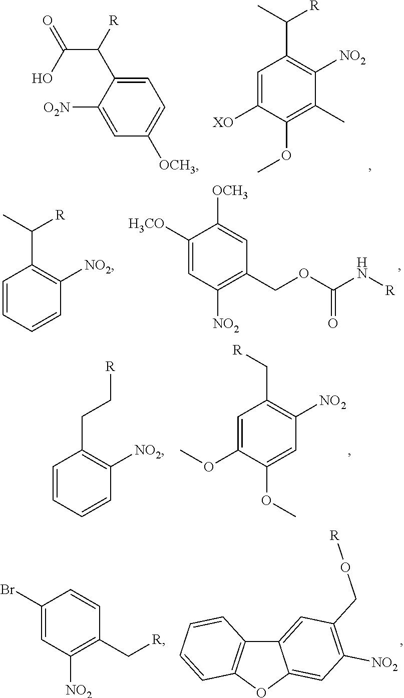

and a combination thereof, wherein each of the structures: X is any halogen group, and R is one of a carboxyl group, an amino group, or a phosphate group, or a combination thereof. As described, individual units of different photodegradable moieties 40 may be incorporated into a single oligonucleotide blocker 38, or individual units of the same photodegradable moiety 40 can be incorporated into a single oligonucleotide blocker 38. The number of individual units of the photodegradable moiety 40 in each nucleotide-based backbone (of each blocker 38) may be any of the numerically-expressed or percentage-based ranges of values disclosed herein. In one example, the percentage of the photodegradable moiety 40 in the nucleotide-based backbone ranges from about 0.01% to about 30% relative to the total number of individual units of the photodegradable moiety plus the total number of individual nucleotides included in the nucleotide-based backbone. In another example, the nucleotide-based backbone includes from 10 nucleotides to 150 nucleotides, and the number of individual units of the photodegradable moiety 40 incorporated into the nucleotide-based backbone ranges from 2 to 10.

The polymeric hydrogel 30 used for the pre-grafted, pre-hybridized polymeric hydrogel 30′ may include any suitable example of the hydrogel materials described herein and may be deposited using any suitable method described herein. A curing process may be performed after the pre-grafted, pre-hybridized polymeric hydrogel 30′ is applied at desired regions of the substrate 14, 16 (e.g., within the lane 22, within depressions 32, or to form functionalized pads 36). The curing process, when performed, may involve exposure of the pre-grafted, pre-hybridized polymeric hydrogel 30′ to energy (e.g., U.V. light, visible light, etc.) or heat.

In some instances, prior to applying the pre-grafted, pre-hybridized polymeric hydrogel 30′ (as shown at B.), this method further comprises activating the portion of the surface of the substrate 14, 16 to introduce surface groups to attach the pre-grafted, pre-hybridized polymeric hydrogel 30′. Activation may involve silanization or plasma ashing of the portion of the substrate surface. Plasma ashing involves the generation of —OH groups at a surface via exposure of the surface to oxygen plasma. Silanization involves the application of a silane or silane derivative over the surface of the substrate 14 or the layer 20 (of the substrate 16). The selection of the silane or silane derivative may depend, in part, upon the polymeric hydrogel 30 that is to be applied. Some example silane derivatives include a cycloalkene unsaturated moiety, such as norbornene, a norbornene derivative (e.g., a (hetero)norbornene including an oxygen or nitrogen in place of one of the carbon atoms), transcyclooctene, transcyclooctene derivatives, transcyclopentene, transcycloheptene, trans-cyclononene, bicyclo[3.3.1]non-1-ene, bicyclo[4.3.1]dec-1 (9)-ene, bicyclo[4.2.1]non-1(8)-ene, and bicyclo[4.2.1]non-1-ene. Any of these cycloalkenes can be substituted, for example, with an R group, such as hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An example of the norbornene derivative includes [(5-bicyclo[2.2.1]hept-2-enyl)ethyl]trimethoxysilane. Other example silane derivatives include a cycloalkyne unsaturated moiety, such as cyclooctyne, a cyclooctyne derivative, or bicyclononynes (e.g., bicyclo[6.1.0]non-4-yne or derivatives thereof, bicyclo[6.1.0]non-2-yne, or bicyclo[6.1.0]non-3-yne). These cycloalkynes can be substituted with any of the R groups described herein. The method used to apply the silane or silane derivative may vary depending upon the silane or silane derivative that is being used. Examples of suitable silanization methods include vapor deposition (e.g., a YES method), spin coating, or other deposition methods. These methods may silanize the entire substrate 14, 16 surface, or the methods silanize only the portion of the substrate 14, 16 surface that is to be coated with the polymeric hydrogel 30′ (e.g., the portion of the substrate 14, 16 that forms the lane 22).

After the pre-grafted, pre-hybridized polymeric hydrogel 30′ is applied over the substrate 14, 16, this example method then proceeds from B. to C. As shown in C., the pre-grafted, pre-hybridized polymeric hydrogel 30′ may be removed from desired portions of the substrate 14, 16, such as interstitial regions 34 or bonding regions 24. The removal of the pre-grafted, pre-hybridized polymeric hydrogel 30′ from these regions 24, 34 may involve a polishing process. The polishing process may be performed with a chemical slurry (including, e.g., an abrasive, a buffer, a chelating agent, a surfactant, and/or a dispersant) which can remove the pre-grafted, pre-hybridized polymeric hydrogel 30′ from the interstitial regions 34 without deleteriously affecting the underlying substrate 14, 16 or the pre-grafted, pre-hybridized polymeric hydrogel 30′ within the lane 22 (or within the depressions 32). Polishing may also be performed with a solution that does not include the abrasive particles. The polishing process may also be performed using polishing head(s)/pad(s) or other polishing tool(s). As an example, the polishing head may be a Strasbaugh ViPRR II polishing head. The pH at the surface of the substrate 14, 16 may be controlled during the process of removing the polymeric hydrogel 30′ (when polishing is performed) so that the double-stranded entities 29 (i.e., the hybridization of the blockers 38 to the primers 26, 28) remain(s) unaffected in the lane 22 or depression 32.

In some instances, such as when photolithography or sacrificial layer materials is/are utilized during the pre-hybridized, pre-grafted polymeric hydrogel 30′ deposition process (or when selective activation of the substrate 14, 16 is performed), the polymeric hydrogel 30′ is applied only within the lane 22 and not over the interstitial regions 34 or bonding region 24. In these instances, polishing (e.g., the removal step depicted at C.) may not be performed.

Another example method shown in FIG. 4 proceeds from A. to D. (rather than from A. to B.). In this example method, the pre-grafted polymeric hydrogel 30″ that is used is not pre-hybridized with the oligonucleotide blockers 38. Rather, the oligonucleotide blockers 38 are hybridized to the primers 26, 28 of the pre-grafted polymeric hydrogel 30″ after it is deposited over the substrate 14, 16.

As shown in D., the pre-grafted polymeric hydrogel 30″, having the primers 26, 28 grafted thereto, is applied over the substrate 14 (or layer 20 of the substrate 16). The polymeric hydrogel 30 used for the pre-grafted, non-pre-hybridized polymeric hydrogel 30″ may include any suitable example of the hydrogel materials described herein, and may be deposited using any suitable method described herein. A curing process may be performed after the pre-grafted, non-pre-hybridized polymeric hydrogel 30″ is applied at desired regions of the substrate 14, 16 (e.g., within the lane 22 or within depressions 32).

This example method then proceeds from D. to E. As shown in E., the pre-grafted, non-pre-hybridized polymeric hydrogel 30″ is removed from desired portions of the substrate 14 or layer 20, such as interstitial regions 34 or bonding regions 24. Any suitable removal process described herein may be used, such as polishing using a chemical slurry or polishing tools, as described. In some instances, such as when photolithography or sacrificial layer materials is/are utilized during the pre-grafted, non-pre-hybridized polymeric hydrogel 30″ deposition process, the polymeric hydrogel 30″ is applied only within the lane 22 and not over the interstitial regions 34 or bonding region 24. In these instances, polishing (e.g., the removal step depicted at E.) may not be performed.

This example method then proceeds from E. to F. As shown at F., the plurality of photodegradable oligonucleotide blockers 38 is introduced, and individual blockers 38 respectively hybridize to at least some of the individual primers 26, 28 attached to the polymeric hydrogel 30″. As such, in this example method, after attaching the plurality of primers 26, 28 to the polymeric hydrogel 30″ and applying the polymeric hydrogel 30″ over the at least the portion of the surface of the substrate 14, 16, the photodegradable oligonucleotide blockers 38 are respectively hybridized to at least some of the plurality of primers 26, 28.

The introduction of the photodegradable oligonucleotide blockers 38 may involve any suitable deposition method disclosed herein, such as puddle dispensing, flow-through deposition, etc. Once deposited, the photodegradable oligonucleotide blockers 38 are allowed to incubate within the lane 22 (or depression 32) at a suitable temperature to enable hybridization. As described herein, during the incubation process, the nucleotides in the nucleotide-based backbone of the oligonucleotide blockers 38 base-pair (i.e., hybridize) with respective primers 26, 28, and the presence of the hybridized blocker 38 prevents subsequently introduced (adapter-tagged) DNA library templates from seeding to the primers 26, 28 until and if the blockers 38 are removed (e.g., using light exposure).

The example methods shown in FIG. 4 depict the addition of the surface chemistry to the lane 22 (or a single depression 32). When the polymeric hydrogel portion of the surface chemistry in these examples is in the form pads 36, the methods involve B. or D. and F. because pad 36 formation does not involve removal of the polymeric hydrogel (pre-grafted or not) from the interstitial regions 34. When pads 36 are formed, a planar substrate 14, 16 is used that can be patterned with the functionalized pads 36 (similar to the patterned structure 17B in FIG. 1D). In these examples, forming the pads 36 involves depositing the pre-grafted, pre-hybridized polymeric hydrogel 30′ (step B.) or the pre-grafted, non-pre-hybridized polymeric hydrogel 30″ (step D.) using a mask that blocks the pre-grafted hydrogel 30′ or 30″ from being applied to areas that will form the interstitial regions 34 and/or bonding regions 24, or using selective deposition techniques (e.g., via inkjet or microcontact printing) that apply the pre-grafted hydrogel 30′ or 30″ only in areas that will form the pads 36. The other processes of the methods (A. and B. or A., D., and F), such as pre-grafting and/pre-hybridization, are performed as described herein.

Method Utilizing a Non-pre-grafted Polymeric Hydrogel

Two methods are respectively shown in FIG. 5 at A., B., C., D., E. and at A. B., C., E. In either of the two methods shown in FIG. 5, the polymeric hydrogel 30 that is used is a non-pre-grafted polymeric hydrogel 30, meaning that the primers 26, 28 are/become attached to the polymeric hydrogel 30 after the hydrogel 30 is deposited over the substrate 14, 16. As such, in either of the two methods shown in FIG. 5, the attaching of the plurality of primers 26, 28 to the polymeric hydrogel 30 is performed after applying the polymeric hydrogel 30 over the at least the portion of the surface of the substrate 14, 16.

As shown in FIG. 5 at A., the substrate 14 (or the layer 20 of the substrate 16) has a lane 22 defined therein (similar to the unpatterned structure 15 depicted in FIG. 1B). It is to be understood, however, that the example methods shown in FIG. 5 may alternatively utilize the patterned structure 17A shown in FIG. 1C (including depressions 32). In any case, either the single layer substrate 14 or the multi-layer substrate 16 including the base support 18 and the layer 20 may be used.

The lane 22 may be defined in the substrate 14 (or in the layer 20 of the substrate 16) using any suitable technique described herein (e.g., etching, nanoimprint lithography, photolithography, etc.). The material of the substrate 14 or the material of the components of the substrate 16 (i.e., the material of the base support 18 and the layer 20) may be any suitable example set forth herein. The patterning technique that is used for the substrate 14 or the layer 20 will depend, in part, upon the material selected for the substrate 14 or the layer 20.

Both of the methods shown in FIG. 5 proceed from A. to B. As shown at B., the non-pre-grafted polymeric hydrogel 30 is applied over the substrate 14 or over the layer 20 of the substrate 16. The hydrogel used for the non-pre-grafted polymeric hydrogel 30 may include any suitable example of the hydrogel materials described herein and may be deposited using any suitable method described herein. A curing process may be performed after the non-pre-grafted polymeric hydrogel 30 is applied at desired regions of the substrate 14, 16 (e.g., within the lane 22, within depressions 32, or to form functionalized pads 36). The curing process, when performed, may involve exposure of the non-pre-grafted polymeric hydrogel 30 to energy (e.g., U.V. light, visible light, etc.) or heat.

In some instances, prior to applying the polymeric hydrogel 30 at B., the methods shown in FIG. 5 further comprise activating the portion of the surface of the substrate 14, 16 to introduce surface groups to attach the polymeric hydrogel 30. Activation may involve silanization or plasma ashing of the entire surface of the substrate 14, 16, or silanization/plasma ashing of just the portion of the substrate 14, 16 where the polymeric hydrogel 30 is to be applied.

Both example methods shown in FIG. 5 then proceed from B. to C. As shown at C., the polymeric hydrogel 30 may be removed from desired portions of the substrate 14, 16, such as interstitial regions 34 or bonding regions 24. The removal of the polymeric hydrogel 30 from these regions 24, 34 may involve the polishing process described herein. In some instances, such as when photolithography or sacrificial layer materials is/are utilized during the non-pre-grafted polymeric hydrogel 30 deposition process, the polymeric hydrogel 30 is applied only within the lane 22 and not over the interstitial regions 34 or bonding region(s) 24. In these instances, polishing (e.g., the removal step depicted at C.) may not be performed.

One of the example methods shown in FIG. 5 proceeds from C. to D. As shown at D., the plurality of primers 26, 28 are introduced to the hydrogel-coated surface of the substrate 14, 16, such that the primers 26, 28 become attached to the polymeric hydrogel 30 within the lane 22 (or to the hydrogel 30 within the depressions 32, not shown). The primers 26, 28 may include any example of the P5, P7, P15, and PA-PD primers 26, 28 disclosed herein, and the primers 26, 28 may be attached/grafted to the polymeric hydrogel 30 using any suitable deposition technique disclosed herein. In this example, the primers 26, 28 that are introduced are not respectively pre-hybridized to individual blockers 38 (of the plurality of photodegradable oligonucleotide blockers 38).

After the non-pre-grafted, non-pre-hybridized polymeric hydrogel 30 has been grafted with the primers 26, 28, this example method then proceeds from D. to E. As shown at E., the plurality of photodegradable oligonucleotide blockers 38 is introduced to the polymeric hydrogel 30 within the lane 22, and individual blockers 38 hybridize to respective primers 26, 28 attached to the polymeric hydrogel 30. As such, in this example, after attaching the plurality of primers 26, 28 to the polymeric hydrogel 30, the photodegradable oligonucleotide blockers 38 are respectively hybridized to the at least some of the plurality of primers 26, 28. The photodegradable oligonucleotide blockers 38 may include any suitable photodegradable moiety 40 (or moieties 40) described herein, and the blockers 38 may be introduced using any suitable deposition technique disclosed herein. The blockers 38 are allowed to incubate as described herein such that they hybridize to respective primers 26, 28.

Another example method shown in FIG. 5 proceeds directly from C. to E. (rather than from C. to D.). In this example, the primers 26, 28 that are introduced are respectively pre-hybridized to individual blockers 38 (of the plurality of photodegradable oligonucleotide blockers 38), which forms a plurality of double stranded entities 29. As such, in the example method that proceeds from C. to E. in FIG. 5, prior to attaching the plurality of primers 26, 28 to the polymeric hydrogel 30, the photodegradable oligonucleotide blockers 38 are respectively hybridized to the at least some of the plurality of primers 26, 28 to form a plurality of double stranded entities 29; and attaching the plurality of primers 26, 28 to the polymeric hydrogel involves attaching the double stranded entities 29 to the polymeric hydrogel 30.

The hybridization of the plurality of primers 26, 28 to the photodegradable oligonucleotide blockers 38 (and the subsequent formation of the plurality of double stranded entities 29) may involve incubating the primers 26, 28 and the blockers 38 in a separate solution at a suitable hybridization temperature, and then introducing the solution to the polymeric hydrogel-coated substrate 14, 16 surface. The attachment of the plurality of double-stranded entities 29 to the polymeric hydrogel 30 may be achieved during this process, and the process may involve any suitable deposition technique disclosed herein. The double-stranded entities 29 may be allowed to incubate for the desired attachment to the polymeric hydrogel 30 to take place. The presence of the hybridized blocker 38 (forming the double stranded entities 29) prevents subsequently introduced (adapter-tagged) DNA library templates from seeding to the primers 26, 28 until/if the blockers 38 are removed (e.g., using light exposure).

The example methods shown in FIG. 5 depict the addition of the surface chemistry to the lane 22 (or a single depression 32). When the polymeric hydrogel 30 portion of the surface chemistry in these examples is in the form pads 36, the methods involve B., D. and E. or B., and E. because pad formation does not involve removal of the polymeric hydrogel 30 from the interstitial regions 34. When pads 36 are formed, a planar substrate 14, 16 is used that can be patterned with the functionalized pads 36 (similar to the patterned structure 17B in FIG. 1D). In these examples, forming the pads 36 involves depositing the non-pre-grafted polymeric hydrogel 30 (step B.) using a mask that blocks the hydrogel 30 from being applied to areas that will form the interstitial regions 34 and/or bonding regions 24, or using selective deposition techniques (e.g., via inkjet or microcontact printing) that apply the non-pre-grafted hydrogel 30 only in areas that will form the pads 36. The other processes of the methods (D. and E., or E.), such as grafting and hybridization, are performed as described herein.

### Method of Using the Flow Cell

A method of using flow cells 10, 10′ including photodegradable oligonucleotide blockers 38 that are hybridized to individual primers 26, 28 will now be described. This method may be used regardless of the method(s) that is/are used to position the surface chemistry (i.e., the polymeric hydrogel 30, the primers 26, 28, and the plurality of photodegradable oligonucleotide blockers 38) at desired regions of the substrate 14, 16.

A method of using a flow cell 10 (or 10′) that includes the plurality of photodegradable oligonucleotide blockers 38 is shown in FIG. 6A through FIG. 6D. For ease of illustration, the flow cell 10 shown in FIG. 6A through FIG. 6D is an open-wafer version of the flow cell 10. It is to be understood, however, that enclosed versions of the flow cell 10 may be used (e.g., versions that include the (un)patterned structure 15, 17A, 17B bonded to the lid or that include two (un)patterned structures 15, 17A, 17B bonded together). Further, while FIG. 6A through FIG. 6D depict an example of a method utilizing a flow cell 10 including the substrate 14, 16 that has the lane 22 defined therein (similar to the unpatterned structure 15 depicted in FIG. 1B), the method may alternatively utilize the patterned structure 17A shown in FIG. 1C (including depressions 32) or the patterned structure 17B shown in FIG. 1D (including functionalized pads 36). Still further, while FIG. 6A through FIG. 6D depict an example of a method utilizing a flow cell 10 (e.g., a flow cell that does not include the CMOS chip 94) it is to be understood that this method is applicable to flow cells 10′ (including the CMOS chip 94 coupled thereto) as well. In any case, either the single layer substrate 14 or the multi-layer substrate 16 including the base support 18 and the layer 20 may be used.

The method shown in FIG. 6A through FIG. 6D generally involves exposing a first sub-set 42A of the plurality of primers 26, 28 to a first preselected wavelength of light, thereby removing the photodegradable oligonucleotide blockers 38 from the primers 26, 28 of the first sub-set 42A; introducing library templates 44 from a first DNA sample to the flow cell 10, thereby respectively seeding at least some of the library templates 44 to at least some of the primers 26, 28 of the first sub-set 42A; and introducing a second plurality of photodegradable oligonucleotide blockers 38′ to hybridize unseeded primers 26′, 28′ of the first sub-set 42A. At the outset of the method shown in FIG. 6A through FIG. 6D, the hybridized primers 26, 28 and blockers 38 are illustrated as and sometimes referred to as the double-stranded entities 29.

FIG. 6A depicts a flow cell 10, where the substrate 14 (or layer 20 of the substrate 16) has the lane 22 defined therein. The lane 22 includes surface chemistry (e.g., the polymeric hydrogel 30, the primers 26, 28 attached to the polymeric hydrogel 30, and the plurality of photodegradable oligonucleotide blockers 38 respectively hybridized to individual primers 26, 28). The structure shown in FIG. 6A may be generated using any suitable technique disclosed herein, such as those described in regard to FIG. 4 and/or FIG. 5. Further, the polymeric hydrogel 30 may include any hydrogel material described herein. Still further, the primers 26, 28 may be any example of the P5, P7, P15, and PA-PD primers 26, 28 disclosed herein. Yet further, the plurality of oligonucleotide blockers 38 may include any suitable photodegradable moiety 40 (or moieties 40) disclosed herein.

As shown in FIG. 6B, a first sub-set 42A of the plurality of primers 26, 28 is exposed to the first preselected wavelength of light (represented by hv in FIG. 6B). In other words, the double-stranded entities 29 whose primers 26, 28 make up the first sub-set 42 are exposed to the first preselected wavelength of light. A dosage of the first preselected wavelength of light hv may be delivered to the first sub-set 42A using a suitable excitation energy (e.g., light) source, such as a UV light source. The light source may be a narrow band light source (e.g., a laser), or a broad band light source depending, in part, on whether the sub-sets 42A, 42B are susceptible to the same wavelength(s) of light and/or whether a mask is used to selectively block some of the light. The first preselected wavelength of light hv that is used will depend, in part, upon the chemical structure of photodegradable moiety 40 (or moieties 40) included in the nucleotide-based backbone of each of the photodegradable oligonucleotide blockers 38. In an example, the first preselected wavelength of light hv ranges from about 300 nm to about 450 nm. The exposure of the double-stranded entities 29 in the first sub-set 42A to the first preselected wavelength of light hv results in the degradation/transformation of the photodegradable moiety 40 (or moieties 40) of the blocker(s) 38 attached to the primers 26, 28 in the first sub-set 42A. This degradation/transformation of the photodegradable moiety 40 (or moieties 40) removes the blockers 38 from the primers 26, 28 in the first sub-set 42A (or renders the blockers 38 hybridized to the primers 26, 28 in first the sub-set 42A susceptible to removal conditions). However, in some examples and as shown in FIG. 6B, the blockers 38 hybridized to the primers 26, 28 (i.e., the double-stranded entities 29) in a second sub-set 42B are not exposed to the preselected wavelength of light hv. In other examples, the double-stranded entities 29 in the second sub-set 42B are exposed to the first preselected wavelength of light hv but do not react to the first preselected wavelength of light hv. In either of these examples, the primers 26, 28 in the second sub-set 42B remain blocked/passivated by the blockers 38 (and thus will not seed subsequently introduced DNA library templates 44 thereto). After the blockers 38 are exposed to the preselected wavelength of light hv, a washing step may be performed using a suitable fluid (e.g., water).

As shown in FIG. 6C, DNA library templates 44 may then be introduced into the lane 22, whereupon individual library templates 44 seed to at least some of the primers 26, 28 in the first sub-set 42A. During the seeding process, the individual library templates 44 (which may be tagged with adapter sequences) hybridize to de-blocked primers 26, 28 in the first sub-set 42A. As further shown in FIG. 6C, in some instances, at least some of the de-blocked primers 26′, 28′ in the first sub-set 42A remain unseeded (e.g., do not seed a DNA library template 44 thereto). These primers 26′, 28′ are referred to as “unseeded primers” of the first sub-set 42A.

As shown in FIG. 6D, the second plurality of photodegradable oligonucleotide blockers 38′ is then introduced into the lane 22, such that the second plurality of photodegradable oligonucleotide blockers 38′ hybridizes to unseeded primers 26′, 28′ of the first sub-set 42A. The second plurality of photodegradable oligonucleotide blockers 38′ may be the same as the first plurality of oligonucleotide blockers 38, or the blockers 38, 38′ may be different (e.g., may include different photodegradable moieties 40, different numbers of nucleotides in the nucleotide-based backbone, different configurations of the photodegradable moiety 40 and the nucleotides in the backbone, etc.) as long as the blockers 38′ can hybridize to the unseeded primers 26′, 28′. An incubation process (performed at a suitable temperature for a suitable period of time) may be performed after the second plurality of photodegradable oligonucleotide blockers 38′ is introduced, during which the blockers 38′ hybridize to the unseeded primers 26′, 28′ in the first sub-set 42A.

The process described in regard to FIG. 6A through FIG. 6D may be repeated for individual sub-sets 42B, etc. of the primers 26, 28, such as the sub-set 42B shown in FIG. 6A through FIG. 6D and/or for any additional sub-sets. Each sub-set 42B, etc. of primers 26, 28 (having blockers 38 hybridized thereto) may respectively be de-blocked using a preselected light wavelength, seeded with DNA library templates 44 from an individual DNA library, and then re-blocked using a second plurality of photodegradable oligonucleotide blockers 38′. Because the photodegradable oligonucleotide blockers 38 prevent the seeding of DNA library templates 44 to a given sub-set 42A, 42B of primers 26, 28 until that particular sub-set 42A, 42B is ready to be used, the method shown in FIG. 6A through FIG. 6D allows templates 44 from two or more different DNA libraries (and thus, different DNA samples) to be respectively seeded to different sub-sets 42A, 42B of primers 26, 28 within discrete regions of the flow cell 10 (or flow cell 10′). It is to be understood that the additional cycles that are performed may be performed in a sequential manner such that different sub-sets 42B, etc. are unblocked, exposed to a different DNA library, and then reblocked, until each sub-set 42A, 42B, etc. is seeded.

When all of the library templates 44 are seeded, all of the photodegradable oligonucleotide blockers 38′ are removed, via an appropriate method as described herein, to expose the primers 26, 28 for use in amplification.

After the desired number of DNA libraries is selectively seeded within distinct regions of the substrate 14, 16 surface and the photodegradable oligonucleotide blockers 38′ are removed, amplification processes and biological sequencing operations may be performed.

In an example, the sequencing operation involves sequencing by synthesis. In sequencing by synthesis, amplification involves cluster generation. In one example of cluster generation, the seeded library templates 44 are copied from the hybridized primers 26, 28 by 3′ extension using a high-fidelity DNA polymerase. The original library templates 44 are denatured, leaving the copies immobilized in the lane 22, in the depressions 32, or over the functionalized pads 36. Isothermal bridge amplification or some other form of amplification may be used to amplify the immobilized copies. For example, the copied templates loop over to hybridize to an adjacent, complementary primer 26, 28, and a polymerase copies the copied templates to form double stranded bridges, which are denatured to form two single stranded strands. These two strands loop over and hybridize to adjacent, complementary primers 26, 28 and are extended again to form two new double stranded loops. The process is repeated on each template copy by cycles of isothermal denaturation and amplification to create dense clonal clusters in the lane 22, in the depressions 32, or over the functionalized pads 36. Each cluster of double stranded bridges is denatured. In an example, the reverse strand is removed by cleaving at the cleavage site (e.g., specific base cleavage), leaving forward template strands. In another example, the forward strand is removed by cleaving at the cleavage site, leaving reverse template strands. Clustering results in the formation of several different template strand copies immobilized in different regions of the flow cell 10, 10′. The clusters in a given region will depend upon the sub-sets 42A, 42B that are used during seeding. This example of clustering is referred to as bridge amplification, and is one example of the amplification that may be performed. It is to be understood that other amplification techniques may be used, e.g., exclusion amplification.

Sequencing primers may then be introduced to the flow cell 10, 10′. The sequencing primers hybridize to a complementary portion of the sequence of the template strand copies that are attached to the lane 22, in the depressions 32, or over the functionalized pads 36. The sequencing primers render the template strand copies ready for sequencing.

An incorporation mix including labeled nucleotides may then be introduced into the flow cell 10, 10′ e.g., via the inlet. In addition to the labeled nucleotides, the incorporation mix may include water, a buffer, and polymerases capable of nucleotide incorporation. When the incorporation mix is introduced into the flow cell 10, 10′, the mix enters the flow channel 12, and contacts the template strand copies.

The incorporation mix is allowed to incubate in the flow cell 10, 10′, and labeled nucleotides (including optical labels) are incorporated by respective polymerases into the nascent strands along the template strand copies. During incorporation, one of the labeled nucleotides is incorporated, by a respective polymerase, into one nascent strand that extends one sequencing primer and that is complementary to one of the template strand copies. Incorporation is performed in a template strand dependent fashion, and thus detection of the order and type of labeled nucleotides added to the nascent strand can be used to determine the sequence of the template strand copies. Incorporation occurs in at least some of the template strand copies during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 10, 10′ during a wash cycle. The wash cycle may involve a flow-through technique, where a washing solution (e.g., buffer) is directed into, through, and then out of flow channel 12, e.g., by a pump or other suitable mechanism.

Without further incorporation taking place, the most recently incorporated labeled nucleotides can be detected through an imaging event. During the imaging event, an illumination system may provide an excitation light to the flow cell 10, 10′. The optical labels of the incorporated labeled nucleotides emit optical signals in response to the excitation light.

After imaging is performed, a cleavage mix may then be introduced into the flow cell 10, 10′. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strand copies are sequenced. It is to be understood that the DNA samples from which the different library templates 44 are generated, and whose copies are sequenced, can be identified, in part, by the respective region of the flow cell 10, 10′ at which the library templates 44 seed. This technique provides a form of indexing of the different DNA samples.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. A flow cell, comprising:
a substrate;
a polymeric hydrogel applied over at least a portion of a surface of the substrate;
a plurality of primers attached to the polymeric hydrogel; and
a plurality of photodegradable oligonucleotide blockers respectively hybridized to at least some of the plurality of primers.

2. The flow cell as defined in claim 1, wherein each of the plurality of photodegradable oligonucleotide blockers includes a nucleotide-based backbone having a photodegradable moiety incorporated therein.

3. The flow cell as defined in claim 2, wherein the photodegradable moiety incorporated into the nucleotide-based backbone is an ortho-nitrobenzyl containing moiety.

4. The flow cell as defined in claim 3, wherein the ortho-nitrobenzyl containing moiety has the following structure: 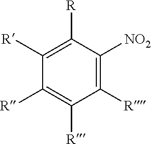
where each of R, R′, R″, R″′, and R″″ is independently selected from the group consisting of a hydrogen, a halogen, an alcohol, an ether, an ester, and a linear or branched alkyl including a terminal carboxyl group, a terminal amino group, or a terminal phosphate group.

5. The flow cell as defined in claim 3, wherein the ortho-nitrobenzyl containing moiety is selected from the group consisting of: 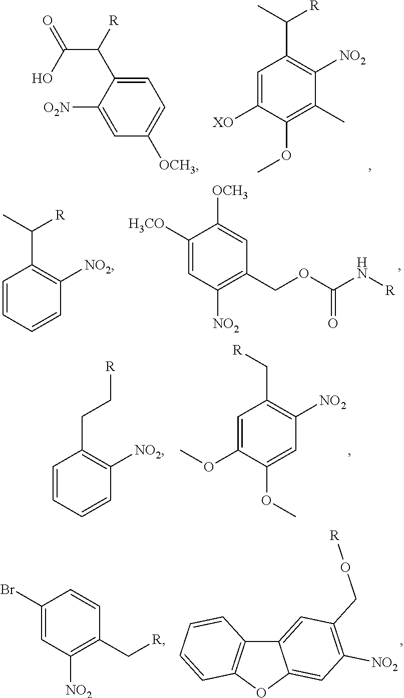
and a combination thereof.

6. The flow cell as defined in claim 2, wherein the photodegradable moiety incorporated into the nucleotide-based backbone is photodegradable at a light wavelength ranging from about 300 nm to about 450 nm.

7. The flow cell as defined in claim 2, wherein a number of individual units of the photodegradable moiety incorporated into the nucleotide-based backbone ranges from 2 to 10.

8. The flow cell as defined in claim 2, wherein a percentage of the photodegradable moiety incorporated into the nucleotide-based backbone ranges from about 0.01% to about 30% relative to a total number of individual units of the photodegradable moiety plus a total number of individual nucleotides included in the nucleotide-based backbone.

9. The flow cell as defined in claim 8 wherein:
the nucleotide-based backbone includes from 10 nucleotides to 150 nucleotides; and
a number of individual units of the photodegradable moiety incorporated into the nucleotide-based backbone ranges from 2 to 10.

10. The flow cell as defined in claim 1, wherein each of the plurality of primers includes a linker, and wherein the each of the plurality of primers is respectively attached to the polymeric hydrogel via the linker.

11. The flow cell as defined in claim 1, further comprising a plurality of depressions defined in the substrate, and wherein the polymeric hydrogel is applied within each of the plurality of depressions.

12. A method of using the flow cell of claim 1, the method comprising:
exposing a first sub-set of the plurality of primers to a first preselected wavelength of light, thereby removing the photodegradable oligonucleotide blockers from the primers of the first sub-set;
introducing library templates from a first DNA sample to the flow cell, thereby respectively seeding at least some of the library templates to at least some of the primers of the first sub-set; and
introducing a second plurality of photodegradable oligonucleotide blockers to hybridize unseeded primers of the first sub-set.

13. The method as defined in claim 12, wherein the first preselected wavelength of light ranges from about 300 nm to about 450 nm.

14. A method, comprising:
applying a polymeric hydrogel over at least a portion of a surface of a substrate;
attaching a plurality of primers to the polymeric hydrogel; and
hybridizing a plurality of photodegradable oligonucleotide blockers to at least some of the plurality of primers.

15. The method as defined in claim 14, wherein each of the plurality of photodegradable oligonucleotide blockers includes a nucleotide-based backbone having a photodegradable moiety incorporated therein.

16. The method as defined in claim 15, wherein the photodegradable moiety incorporated into the nucleotide-based backbone is an ortho-nitrobenzyl containing moiety.

17. The method as defined in claim 16, wherein the ortho-nitrobenzyl containing moiety has the following structure: 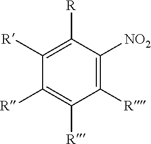
where each of R, R′, R″, R″′, and R″″ is independently selected from the group consisting of a hydrogen, a halogen, an alcohol, an ether, an ester, and a linear or branched alkyl including a terminal carboxyl group, a terminal amino group, or a terminal phosphate group.

18. The method as defined in claim 15, wherein a percentage of the photodegradable moiety in the nucleotide-based backbone ranges from about 0.01% to about 30% relative to a total number of individual units of the photodegradable moiety plus a total number of individual nucleotides included in the nucleotide-based backbone.

19. The method as defined in claim 15 wherein:
the nucleotide-based backbone includes from 10 nucleotides to 150 nucleotides; and
a number of individual units of the photodegradable moiety incorporated into the nucleotide-based backbone ranges from 2 to 10.

20. The method as defined in claim 14, wherein prior to applying the polymeric hydrogel, the method further comprises activating the portion of the surface of the substrate to introduce surface groups to attach the polymeric hydrogel.

21. The method as defined in claim 14, wherein the attaching of the plurality of primers to the polymeric hydrogel is performed prior to applying the polymeric hydrogel over the at least the portion of the surface of the substrate.

22. The method as defined in claim 21, wherein:
prior to attaching the plurality of primers to the polymeric hydrogel, the photodegradable oligonucleotide blockers are respectively hybridized to the at least some of the plurality of primers to form a plurality of double stranded entities; and
attaching the plurality of primers to the polymeric hydrogel involves attaching the plurality of double stranded entities to the polymeric hydrogel.

23. The method as defined in claim 21, wherein after attaching the plurality of primers to the polymeric hydrogel and applying the polymeric hydrogel over the at least the portion of the surface of the substrate, the photodegradable oligonucleotide blockers are respectively hybridized to the at least some of the plurality of primers.

24. The method as defined in claim 14, wherein the attaching of the plurality of primers is performed after applying the polymeric hydrogel over the at least the portion of the surface of the substrate.

25. The method as defined in claim 24, wherein:
prior to attaching the plurality of primers to the polymeric hydrogel, the photodegradable oligonucleotide blockers are respectively hybridized to the at least some of the plurality of primers to form a plurality of double stranded entities; and
attaching the plurality of primers to the polymeric hydrogel involves attaching the double stranded entities to the polymeric hydrogel.

26. The method as defined in claim 24, wherein after attaching the plurality of primers to the polymeric hydrogel, the photodegradable oligonucleotide blockers are respectively hybridized to the at least some of the plurality of primers.
